(19) United States
(12) Patent Application Publication
(10) Pub. No.: US 2024/0325760 A1
(43) Pub. Date: Oct. 3, 2024

(54) METHODS FOR DETECTING AND PREVENTING ALZHEIMER'S DISEASE AND MILD COGNITIVE IMPAIRMENT

(71) Applicant: RAMOT AT TEL-AVIV UNIVERSITY LTD., Tel Aviv (IL)

(72) Inventors: Inna SLUTSKY, Tel Aviv (IL); Shiri SHOOB, Tel Aviv (IL); Nadav BUCHBINDER, Tel Aviv (IL); Daniel ZARHIN, Tel Aviv (IL)

(21) Appl. No.: 18/742,987

(22) Filed: Jun. 13, 2024

Related U.S. Application Data
(63) Continuation-in-part of application No. PCT/IL2022/051323, filed on Dec. 14, 2022.
(60) Provisional application No. 63/289,886, filed on Dec. 15, 2021.

(51) Int. Cl. A61N 1/36; A61B 5/00; A61B 5/055; A61B 5/245; A61B 5/374; A61K 45/06

(52) U.S. Cl. CPC A61N 1/36135; A61B 5/055; A61B 5/245; A61B 5/374; A61B 5/4088; A61B 5/7275; A61K 45/06; A61N 1/36082; A61N 1/36146

(57) ABSTRACT

Methods are for determining whether a subject who does not present clinical symptoms associated with Alzheimer's disease (AD) or mild cognitive impairment (MCI) is in a process of developing AD or MCI, or at risk for developing post-anesthesia delirium or cognitive dysfunction, by detecting the presence of epileptiform spikes in the brain of the subject during diagnostic anesthesia. Additional methods are for preventing, reducing likelihood of, or treating AD or MCI, or post-anesthesia delirium or cognitive dysfunction.

### CROSS-REFERENCE TO RELATED APPLICATIONS

The present application is a continuation-in-part of International Application No. PCT/IL2022/051323, filed Dec. 14, 2022, designating the U.S., and published as WO 2023/112030 on Jun. 22, 2023, which claims the benefit of U.S. Provisional Application No. 63/289,886, filed Dec. 15, 2021. Any and all applications for which a foreign or domestic priority claim is identified above and/or in the Application Data Sheet as filed with the present application are hereby incorporated by reference under 37 CFR 1.57, the entire contents of each and all these applications being herewith incorporated by reference in their entirety as if fully disclosed herein.

### FIELD OF THE INVENTION

The present invention relates in general to methods for diagnosing and preventing Alzheimer's disease (AD) and/or mild cognitive impairment (MCI), as well as post-operative delirium or cognitive dysfunction.

### BACKGROUND OF THE INVENTION

Alzheimer's disease (AD) is a progressive neurodegenerative disorder accounting for the vast majority of dementia. Hippocampal and cortical circuit dysfunctions are hypothesized to cause cognitive deficits in AD such as episodic and spatial memory impairments. However, the onset of amyloid-β (Aβ) depositions precedes cognitive impairments by at least 10-20 years, marking a significant pre-symptomatic disease stage. By the time the earliest AD clinical symptoms are detectable, Aβ accumulation is close to reaching its peak, followed by intracellular aggregation of tau. This suggests that homeostatic mechanisms successfully maintain critical aspects of neural circuits during early impairments of Aβ and tau proteostasis, but fail at some point, driving the emergence of the first symptoms. Identifying how circuit-level signatures are altered during the pre-symptomatic AD stage is crucial for understanding the transition from ‘silent’ pathophysiology to clinically evident impairments.

Mild cognitive impairment (MCI) is a neurocognitive disorder which involves cognitive impairments beyond those expected based on an individual's age and education, but which are not significant enough to interfere with instrumental activities of daily living. MCI may occur as a transitional stage between normal aging and dementia, especially AD.

Extensive experimental efforts over the past decades have identified the role of familial AD (fAD) mutations in early impairments of synaptic transmission and plasticity in hippocampal circuits via Aβ and other cleavage products of amyloid precursor protein (APP) processing. In addition to synaptic plasticity deficits, emerging evidence points to hyperexcitability of hippocampal and cortical neural networks in patients with amnestic MCI (Bakker et al., 2012, Lam et al., 2017, Quiroz et al., 2010; Vossel et al., 2013; Vossel et al., 2016), and in distinct fAD mouse models (Busche et al., 2012; Busche et al., 2008; Hall et al., 2015; Minkeviciene et al., 2009; Palop et al., 2007; Palop and Mucke, 2009; Verret et al., 2012).

It is hypothesized that homeostatic plasticity bi-directionally regulates neuronal activity around a stable set point to compensate for learning-related plasticity. Emerging evidence suggests that the distribution of firing rates amongst neurons in a neuronal circuit and their mean firing rate (MFR) are the key variables that are maintained around a set-point value in a process called firing rate homeostasis. A wide repertoire of homeostatic plasticity mechanisms stabilizing MFR set points have been identified, including changes in synaptic strength, intrinsic excitability, and excitation-to-inhibition ratio. Our recent study suggests that homeostatic MFR set points are not predetermined, but can be tuned by readjusting the compensatory feedback mechanisms to maintain a distinct MFR set-point value (Styr et al., 2019). Indeed, MFRs are physiologically regulated by arousal states in distinct neural circuits. MFRs decrease during sleep, but return to higher MFR set points following transitions into active wakefulness in the rodent hippocampus and some areas of the cortex. Other parameters of firing rate statistics are also regulated by sleep. For example, non-rapid eye movement (NREM) sleep, which makes up ˜80% of all sleep, is associated with homogenization of firing rate distributions, differentially regulating MFR of high-firing and low-firing rate neurons. These state-dependent changes in firing rate distributions have been proposed to undergo homeostatic regulation (Watson et al., 2016). Interestingly, downward MFR homeostasis in response to hyperactivity occurs during sleep but not wake states in the V1 cortex. Moreover, firing rate homeostasis is locally regulated in neural circuits. For example, MFR is decreased in deep, but not superficial, cortical layers during NREM sleep in the V1 cortex.

State-dependent changes in firing rates may be important for early progression of AD pathology. Aβ and tau soluble protein levels and aggregates are influenced by the sleep-wake cycle (Musiek and Holtzman, 2016; Roh et al., 2012): their levels in the interstitial fluid are decreased by sleep and increased by sleep deprivation (Holth et al., 2019; Ju et al., 2017; Kang et al., 2009; Lucey et al., 2018; Xie et al., 2013). Furthermore, sleep is progressively deteriorated in AD patients and mouse models, resulting in disrupted slow-wave activity (SWA) during NREM sleep, sleep fragmentation and reduction in sleep time (Holth et al., 2017; Luccy et al., 2019; Mander et al., 2015; Musick and Holtzman, 2016; Roh et al., 2012; Wang and Holtzman, 2020).

In addition to natural sleep, general anesthesia leads to a pronounced suppression of MFRs in non-human primates and rodents, as well as to a reduced number of discriminable neural activity patterns (Wenzel et al., 2019). Moreover, distinct general anesthetic drugs augment Aβ and tau soluble protein levels and their aggregation (Berger et al., 2016; Eckenhoff et al., 2004; Planel et al., 2007; Vutskits and Xie, 2016; Whittington et al., 2019; Xie et al., 2008; Zhang et al., 2013). Thus, in addition to sleep, general anesthesia may constitute a distinct low-arousal brain state that could also reveal early firing rate dyshomeostasis.

### SUMMARY OF INVENTION

In one aspect, the present invention provides a method of determining whether a subject is in a process of developing AD or MCI, said method comprising detecting the presence of epileptiform spikes in the brain of said subject during a low-arousal brain state, preferably diagnostic anesthesia, wherein said subject is an asymptomatic subject, i.e., does not present clinical symptoms associated with said AD or MCI; and the presence of epileptiform spikes indicates that said subject is in the process of developing said AD or MCI.

In another aspect, the present invention provides a method of determining whether a subject about to undergo general anesthesia is at risk for developing post-anesthesia delirium or cognitive dysfunction, said method comprising detecting epileptiform spikes in the brain of said subject during a low-arousal brain state, preferably diagnostic anesthesia, wherein said subject does not present clinical symptoms associated with AD or MCI; and the presence of epileptiform spikes in the brain of said subject indicates that said subject is in a process of developing said AD or MCI and thus at risk for developing post-anesthesia delirium or cognitive dysfunction.

In yet another aspect, the present invention provides a method for preventing or delaying the onset of clinical symptoms of AD or MCI in a subject in need thereof, wherein said subject does not present clinical symptoms associated with said AD or MCI, and has been identified as presenting epileptiform spikes in the brain during a low-arousal brain state, preferably diagnostic anesthesia, indicating that said subject is in a process of developing said AD or MCI; and said method comprises providing said subject with a treatment capable of attenuating or reducing cortico-hippocampal hyperactivity, thereby preventing or delaying the onset of clinical symptoms associated with said AD or MCI.

In still another aspect, the present invention provides a method for preventing or attenuating post-anesthesia delirium or cognitive dysfunction in a subject about to undergo general anesthesia, wherein said subject does not present clinical symptoms associated with AD or MCI, and has been identified as presenting epileptiform spikes in the brain during a low-arousal brain state, preferably diagnostic anesthesia, indicating that said subject is in a process of developing AD or MCI; and said method comprises providing said subject, prior to said general anesthesia, with a treatment capable of attenuating or reducing cortico-hippocampal hyperactivity, thereby preventing or attenuating said post-anesthesia delirium or cognitive dysfunction.

In a further aspect, the present invention provides a method of treating AD or MCI in a subject suffering from AD or MCI, i.e., a subject presenting with symptoms associated with AD or with MCI, or preventing or delaying the onset of clinical symptoms associated with said AD or MCI in an asymptomatic subject being in the process of developing AD or MCI, e.g., a subject that has been identified as presenting epileptiform spikes in the brain during a low-arousal brain state, preferably diagnostic anesthesia, said method comprising applying deep brain stimulation (DBS) to the thalamic nRE of the brain of said subject, e.g., a tonic DBS protocol such as a low frequency tonic DBS protocol.

In yet a further aspect, the present invention provides a method of treating post-anesthesia delirium or cognitive dysfunction in a subject in need thereof having undergone general anesthesia, said method comprising providing to said subject a treatment capable of attenuating or reducing cortico-hippocampal hyperactivity, selected from administration of either an active agent capable of reducing DHODH enzyme activity in the CNS or an antiepileptic drug (AED), and application of DBS, preferably to the thalamic nRE, e.g., a tonic DBS protocol such as a low frequency tonic DBS protocol.

In still a further aspect, the present invention provides an active agent capable of reducing DHODH enzyme activity in the CNS, or an AED, for use in preventing or delaying the onset of clinical symptoms associated with AD or MCI in a subject, wherein said subject does not present clinical symptoms associated with said AD or MCI, and has been identified as presenting epileptiform spikes in the brain during a low-arousal brain state, preferably diagnostic anesthesia, indicating that said subject is in a process of developing said AD or MCI.

In yet a further aspect, the present invention provides an active agent capable of reducing DHODH enzyme activity in the CNS, or an AED, for use in preventing or attenuating post-anesthesia delirium or cognitive dysfunction in a subject about to undergo general anesthesia, wherein said subject does not present clinical symptoms associated with AD or MCI, and has been identified as presenting epileptiform spikes in the brain during a low-arousal brain state, preferably diagnostic anesthesia, indicating that said subject is in a process of developing said AD or MCI.

### BRIEF DESCRIPTION OF DRAWINGS

FIGS. 1A-1G show no deficits in the hippocampal region CA1 network activity and CA3-CA1 synaptic transmission during active wakefulness in APP/PS1 mice. (A) Average mean Ca²⁺ event rate (mCaR) distributions of CA1 neuronal populations in WT (6 mice, 3,846 cells, dashed line) and APP/PS1 (6 mice, 3,973 cells, solid line) during active wakefulness. (B-D) No difference was observed for median mCaRs of neurons (mCaR, p=0.94, B), number of active neurons (Nₐ, p=0.99, C) and total activity (mCaR*Nₐ, p=0.82, D) between WT and APP/PS1 mice (same data as in C, averaged per mouse) during active wakefulness; WT—left bars, APP/PS1—right bars. (E) MFRs of regularly spiking neurons during active wakefulness were not different (p=0.82) between in WT (left, 2.01±0.195; 4 mice, 103 single units) and APP/PS1 (right, 1.88±0.15; 4 mice, 98 single units) mice. (F) No changes in CA3-CA1 basal synaptic transmission between awake WT and APP/PS1 mice: field excitatory postsynaptic potential (fEPSP) amplitudes recorded in CA1 in response to increased current injection in the ipsilateral Schaffer Collaterals of awake WT (8 mice) vs. APP/PS1 (6 mice) displayed similar input-output slopes (p=0.19); WT—squares; APP/PS1—circles. (G) No changes in short-term synaptic plasticity between awake WT and APP/PS1 mice: fEPSP amplitudes normalized to the first response during a burst stimulation (five stimuli at 50 Hz) displayed similar levels of synaptic facilitation between behaving WT vs. APP/PS1 (p=0.42); WT—squares; APP/PS1—circles. Unpaired Mann-Whitney nonparametric test (D-F, H). Two-way-ANOVA with Sidak's multiple comparisons test (F-G) were used for the analysis. ns, non-significant. Error bars represent Standard error of the mean (SEM).

FIGS. 2A-2G show that reduction of CA1 population activity during NREM sleep is impaired in APP/PS1 mice. (A-B) Representative raster plots of CA1 Ca²⁺ event rates in active wake-dense episode (A) and NREM-dense episode (B) in a WT mouse. (C-D) Same as A-B for APP/PS1 mice. (E) Average mCaR distributions in CA1 of WT mice (5 mice) during active wake (solid line, 3,319 cells) and NREM sleep (dashed line, 1,741 cells) states. (F) Average mCaR distributions in CA1 of APP/PS1 mice (4 mice) during active wake (solid line, 2,595 cells) and NREM sleep (dashed line, 2,224 cells) states. (G) Relative change in total activity in WT (squares) vs. APP/PS1 mice (circles) by NREM sleep in comparison to active wake state (the same data as in E-F). Two-way ANOVA with Sidak's multiple comparisons test (K—inter-group analysis), two-way ANOVA (K—intra-group analysis). **p<0.01 ns, non-significant. Error bars represent SEM.

FIGS. 3A-3D show that local dysregulation of CA1 firing rates by NREM sleep precedes global SWA deterioration in APP/PS1 mic. (A) NREM sleep (right bar) causes a reduction (p<0.0001) in CA1 MFR of regular spiking neurons from 2.01±0.195 Hz in active wakefulness (AW, left bar) to 1.51±0.12 Hz in WT mice (4 mice, 103 single units). (B) MFR of CA1 regular spiking neurons is not different (p=0.53) between AW (left bar, 1.88±0.15 Hz) and NREM sleep (right bar, 1.91±0.13 Hz) in APP/PS1 mice (4 mice, 98 units). (C) Frontal electroencephalogram (EEG) spectra during NREM sleep in WT (n=5, dashed line) and APP/PS1 (n=5, solid line) mice. Post-hoc comparisons did not reveal significant genotype differences for any of the SWA frequency bins in NREM state (p=0.68). (D) CA1 local field potential (LFP) spectra recorded by the same electrodes as single units in WT (n=4, dashed line) and APP/PS1 (n=4, solid line) mice during NREM sleep. Slow-wave power during NREM is not significantly different between WT and APP/PS1 mice (p=0.14). Non-parametric, paired, two-tailed Wilcoxon test (A,B), two-way ANOVA with Sidak's multiple comparisons test (C,D). ****p<0.0001, ns, non-significant. Error bars represent SEM.

FIGS. 4A-4E show loss of neuronal inhibition across anesthetic depth in APP/PS1 mice. (A-B) Average mCaR distribution of CA1 neuronal populations in WT (A, awake—5 mice, solid line; moderate—5 mice, dashed line; deep—5 mice, dotted line) and APP/PS1 (B, awake—7 mice, solid line; moderate—7 mice, dashed line; deep—4 mice, dotted line) under three behavioral conditions: exploration in familiar environment (Active Wake), under moderate anesthesia (moderate) and under deep anesthesia (deep). Imaging was performed in parallel to LFP recordings in the CA1. Note higher mCaR and number of active neurons in anesthetized APP/PS1 vs. WT mice. (C) Total activity is higher in APP/PS1 vs. WT mice under both, moderate (middle bars) and deep (right bars) anesthesia (same data as in A-B); WT left bar each pair (squares); APP/PS1 right bar each pair (circles). (D-E) Differences in temporal patterns of Ca²⁺ transients in CA1 network of WT (dashed lines) vs. APP/PS1 (solid lines) anesthetized mice. APP/PS1 mice display larger number of cells that participate in network burst (D, p=0.0001 for moderate, upper panel, and deep, lower panel) and larger number of spikes that constitute a network burst (E, p=0.0001 for moderate, upper panel, and deep, lower panel). Two-way-ANOVA with Sidak's multiple comparisons test (C—inter-group analysis,), two-way ANOVA with Tukey's multiple comparison test (D-E), one-way ANOVA with Dunnett's multiple comparisons test (C—intra-group analysis) were used for the analysis. **p<0.01, ***p<0.001, ****p<0.0001, ns, non-significant (p>0.05). Error bars represent SEM.

FIGS. 5A-5B show that CA1 is hyperexcitable under anesthesia in different fAD models. (A) Representative traces of raw LFP recordings using depth electrodes implanted in the CA1, during awake behavior (left) and isoflurane anesthesia (right, moderate anesthesia is shown). Notice the appearance of abnormal spikes under anesthesia. LFP recordings were performed in WT (top) and across different mouse fAD models (top to bottom)—APP/PS1, APP-KI, and 5XFAD. Scale bars: 10 sec, 1 mV. (B) On average, all 3 fAD models displayed higher frequency of abnormal spikes compared to WT across isoflurane-induced moderate (left group, left to right: WT—17 mice, APP/PS1—18 mice, APP-KI—6 mice, FADX5—5 mice) and deep (right group, left to right: WT—22 mice, APP/PS1—22 mice, APP-KI—9 mice, FADX5—8 mice) anesthesia. Two-way-ANOVA with Dunnett's multiple comparisons test (C) was used for the analysis. *p<0.05, **p<0.01 and ****p<0.0001. Error bars represent SEM.

FIGS. 6A-6H show that anesthesia induces CA1 hyperexcitability and transiently impairs spatial working memory and nucleus reuniens (nRE)-CA1 short-term synaptic plasticity in APP/PS1, but not in WT mice. (A) No difference in spatial working memory in delta maze in WT mice (n=9) before (left side filled squares) vs. after (right side empty squares) general anesthesia (GA, p=0.34. Two-way ANOVA with Sidak's multiple comparison tests: 10 sec p=0.27, 20 sec p=0.65, 60 sec p=0.94). (B) Spatial working memory in delta maze is impaired in APP/PS1 mice (n=9) following GA (p=0.0046, two-way ANOVA with Sidak's multiple comparison tests: 10, 20 and 60 sec, p<0.0001); before—left side empty circles; after—right side filled circles. (C) Delta maze success rate of WT (dashed line with squares) and APP-PS1 (solid line with circles) mice before and after GA for the 20 sec delay. The impairment in spatial working memory performance of APP/PS1 mice following GA was transient. Error bars represent SEM. (no difference in the fAD group before vs. 3, 4 or 5 days after GA, using Friedman test with Dann's multiple comparison tests: day −1 or −2 vs. day 3, 4 or 5 p>0.9). (D) Frequency of abnormal CA1 spikes inversely correlates to spatial working memory performance after GA, calculated as the average success rate for 3 days after GA normalized to baseline (Bsl) success rate (average of 3 days before GA), in the 90 seconds delay (Spearman r=−0.9154, p=0.0016). (E, F) The dynamics of paired-pulse facilitation before and after GA in WT (E, n=6) and APP/PS1 (F, n=8) mice. Friedman test with Dann's multiple comparison tests: p=0.0012, 0.0005, and 0.0078 (APP/PS1) and p>0.5 (WT) for the first, second and third day following GA in comparison to the day before GA. (G,H) The dynamic change in the amplitude of fEPSP evoked by the first stimuli of the 25 Hz burst (at 0.03 Hz) in comparison to the baseline before and after GA in WT (G, n=6) and APP/PS1 (H, n=8) mice. Friedman test with Dann's multiple comparison tests: p=0.0047, 0.002, and 0.0072 (APP/PS1) and p>0.8 (WT) for the first, second and third day after GA in comparison to the day before GA. Error bars represent SEM. *p<0.05, **p<0.01, ***p<0.001, ****p<0.0001, ns—non-significant.

FIGS. 7A-7I show that dysregulation of downward MFR homeostasis by fAD mutations in hippocampal networks ex vivo. (A) The MFR of the network was stably reduced after isoflurane (ISO) application to 42.0±0.9% of baseline in WT cultures (n=8 experiments, 633 channels). (B) The MFR of the network was decreased and rapidly compensated to 112.5±8.7% (p>0.9) of baseline after ISO application in APP/PS1 cultures (n=5 experiments, 384 channels). (C) Summary of isoflurane (24 hour) effect on MFRs in WT (left bar) and APP/PS1 (right bar) cultures (same data as A, B). (D-E) A typical MFR homeostatic response to chronic inactivity induced by a GABA(B) receptor agonist baclofen (Bac, 10 μM) in WT (D) and APP/PS1 (E) networks. MFR was homeostatically compensated during the first day of the perturbation to the MFR set-point level in WT (7 experiments, 394 channels, p=0.76) and APP/PS1 (n=6, p=0.64, 377 channels) neurons. (F) Summary of baclofen (48 hr) effect on MFRs in WT (left) and APP/PS1 (right) cultures (same data as D-E). (G-H) MFR was renormalized to the baseline level following 2 days of gabazine (GBZ, 30 μM) application in WT neurons (G, p=0.31, n=6 experiments, 398 channels), but MFR renormalization to GBZ was disrupted in APP/PS1 neurons (H, n=4 experiments, 270 channels). (I) Summary of gabazine (48 hr) effect on MFRs in WT (left) and APP/PS1 (right) cultures (same data as G-H). Paired two-tailed t-test between the baseline and the last 4 hours of the perturbation (A-B, D-E, G-H) and Mann-Whitney nonparametric two-tailed test (E,H,K) were used for the analysis. *p<0.05, **p<0.01, *p<0.001, ns—non-significant. The error bars represent SEM.

FIGS. 8A-8E show that teriflunomide (TERI) reduces CA1 hyperexcitability in anesthetized APP/PS1 mice. (A) Dose-response effect of TERI on the fEPSP amplitude in CA3-CA1 synaptic connections in hippocampal slices of WT (circles, 8 mice, 13 slices) and APP/PS1 (rectangles, 8 mice, 13 slices). IC₅₀: 17.6±1 μM in WT, 19.2±1 μM in APP/PS1. (B) Orotate concentration is not different between hippocampi of anesthetized WT (left bar) and APP/PS1 (right bar) mice (6 WT and 6 APP/PS1 hippocampi, p=0.92). (C) TERI intracerebroventricular (ICV) injection caused a significant reduction from baseline the frequency of high-voltage spikes from 7.6±1.4 to 3.8±0.95 per min (n=9 mice, p=0.004). (D) vehicle (VEH) ICV injections did not affect the frequency of high-voltage spikes (n=7 mice, p=0.58). (E) On average, TERI-treated group displayed 50.7±7.2% reduction (p=0.0003) in the frequency of high-voltage spikes (the same data as in C,D). Wilcoxon matched-pairs (C,D) and Mann-Whitney nonparametric two-tailed test (B,E) were used for the analysis. **p<0.01, ***p<0.001, ns-non-significant. The error bars represent SEM.

FIGS. 9A-9J show bi-directional regulation of CA1 and medial prefrontal cortex (mPFC) hyperexcitability by tonic vs. nRE stimulation under anesthesia in fAD model. (A) Quantification of c-Fos+ cell in the midline thalamus. The amount of cFos+ cells was significantly increased following anesthesia in APP/PS1 (right bar) compared to WT (left bar) mice (p=0.0008, Mann-Whitney test). Scale bar: 100 μm. (B) Local TTX injection (300 nl, 70 μM) to the nRE caused a reduction in the rate of epileptiform spikes in the hippocampal CA1 of anesthetized APP/PS1 mice compared to the baseline (P=0.0156, Wicoxon matched-pair test, n=7). (C) Local injection of CNQX+AP5 (1 μl, CNQX 5 mM+AP5 25 mM) to the nRE caused a reduction in the rate of epileptiform spikes in the hippocampal CA1 of anesthetized APP/PS1 mice (p=0.0039, Wilcoxon matched-pair test, n=9). (D) Rate of CA1 epileptiform spike after VEH (saline) vs. CNO i.p. injections (5 mg/kg) in anesthetized APP/PS1 mice expressing hM4D (Gi)-mCherry in the nRE-CA1 synapses (n=8 mice, p=0.0078, Wilcoxon matched-pairs test). (E) Illustration of stimulation patterns. nRE and SC were stimulated for 400 seconds by either (i) tonic 25 Hz deep brain stimulation (tDBS) or (ii) phasic deep brain stimulation administered as 1.25 s long 100 Hz bursts every 5 s (pDBS). Duration of stimulus pulses was 0.5 ms. (F) Representative traces of CA1 epileptiform spikes in the baseline (top) and after tDBS-nRE (bottom) in anesthetized APP/PS1 mice. Scale bars: 5 mV, 1 min. (G) Representative traces of CA1 epileptiform spikes in the baseline (top) and after pDBS-nRE (bottom) in anesthetized APP/PS1 mice. Scale bars: 5 mV, 1 min. (H) APP/PS1 mice displayed lower frequency of epileptiform CA1 spikes during GA a week after tDBS in the nRE, but not SC (p<0.0001, One-way ANOVA with Sidak's multiple comparison tests: Sham vs. nRE p=<0.0001, nRE vs. SC p=<0.0001, n=6, 9 and 4 for Sham, nRE and SC). (I) APP/PS1 mice displayed higher frequency of epileptiform spikes in CA1 during GA a week after pDBS in the nRE, but not SC (p=0.0002, Kruskal-Wallis test with Dunn's multiple comparison tests: Sham vs. nRE p=0.0326, nRE vs. SC p=0.0060, n=6, 6 and 4 for Sham, nRE and SC). (J) The frequency of epileptiform spikes during GA in the mPFC of APP/PS1 mice was decreased by tDBS and increased by pDBS of the nRE (p<0.0001, One-way ANOVA with Sidak's multiple comparison tests: Sham vs. Tonic p=0.0051, Sham vs. Phasic p=0.0009, n=7, 5 and 4 for Sham, tDBS and pDBS). Error bars represent SEM; *p<0.05, **p<0.01, ****p<0.0001, ns-non-significant.

FIGS. 10A-10E show that tonic nRE stimulation restores anesthesia-induced dyshomeostasis of CA1 activity in APP/PS1 mice. (A) Representative raster plots demonstrating CA1 Ca²⁺ event rates during awake (top) and GA (bottom) states before (left panel), 1 week (middle panel) and 1 month (right panel) after tDBS-nRE. (B) Single-neuron activity (mCaR*Na) during anesthesia, normalized to the activity during awake at the same day, was higher in fAD mice before DBS (middle) compared to WT (left), but after DBS was similar to WT (right, p=0.0394, one-way ANOVA with Sidak's multiple comparison tests: WT vs. fAD p=0.0347, n=3, WT vs. fAD+DBS p=0.9566, n=3). (C-E) Effect of GA on average Ca²⁺ event rate distributions of CA1 neuronal populations in APP/PS1 mice (3 mice, 5,355 cells) before (C), 1 week (D) and 1 month (E) after tDBS-nRE; awake—solid line; anesthetized—dashed line.

FIGS. 11A-11I show that tonic nRE stimulation rescues anesthesia-induced impairments of spatial working memory and short-term synaptic plasticity in APP/PS1 mice. (A) Temporal dynamics of change in spatial working memory performance tested by delta maze of APP/PS1 mice over 3 days of baseline and 3 days following GA, before (solid line with circles) and after (dashed line with triangles) tDBS-nRE in APP-PS1 mice for 60 sec delay (n=8). (B) GA-induced impairment of spatial working memory in delta maze was rescued by tDBS-nRE preceding isoflurane exposure (P=0.0001, mixed effect analysis with Sidak's multiple comparison tests: 60 sec delay-baseline vs. 1^{st} GA: p=0.0021, GA1 vs. GA2: p=0.0306, baseline vs. 2^{nd} GA: p=0.7189; n=8. 90 sec delay-baseline vs. GA1: p=0.0040, GA1 vs. GA2: p=0.0005, baseline vs. 2^{nd} GA: p=0.7774; n=7); in each group of bars: baseline (bsl)—left, untreated (GA)—middle, tDBS treatment (tDBA+GA)—right. (C) Temporal dynamics of change in spatial working memory performance of APP/PS1 mice over 3 days of baseline and 4 days following GA, for the 1^{st} (GA1—solid line with circles) and 2^{nd} (GA2—dashed line with triangles) GA session in APP-PS1 mice for 60 sec delay (n=6). (D) GA-induced impairments of spatial working memory in delta maze, following a second round of anesthesia in the same mice, were similar to the first anesthesia (P=0.0001, mixed effect analysis with Sidak's multiple comparison tests: 60 sec delay-baseline vs. 1^{st} GA: p=0.1104, GA1 vs. GA2: p=0.1547, baseline vs. 2^{nd} GA: p=0.0114; n=6. 90 sec delay-baseline vs. GA1: p=0.0114, GA1 vs. GA2: p=0.1051, baseline vs. 2^{nd} GA: p=0.0004; n=6); in each group of bars: baseline (bsl)—left, GA1—middle, GA2—right. (E-F) Mice tested as described in (A) displayed lower rate of abnormal spikes in the CA1 (E, p=0.0078, paired Wilcoxon test, n=8) and in the mPFC (F, p=0.0469, paired Wilcoxon test, n=7) before and after tDBS-nRE during GA. (G) Burst-suppression ratio (BSR) was not different before and after tDBS-nRE (n=7, p=0.9669, Wilcoxon matched-paired test). (H) Paired-pulse ratio in the nRE-CA1 synapse of WT (n=5), APP/PS1 (n=9) and APP/PS1 after tDBS-nRE (n=5) in the 3 days following GA, normalized to baseline. (p=0.0001, one-way ANOVA with Sidak's multiple comparison tests: WT vs. APP/PS1 p=0.0004, APP/PS1+1DBS vs. APP/PS1 p=0.0011, WT vs. APP/PS1+tDBS p=0.9522). (I) Paired-pulse ratio of fEPSP amplitudes in the nRE-CA1 pathway of awake APP/PS1 mice during baseline and the 4 days following GA (1.5% isoflurane for 3 hours) before (solid line with filled circles) and after tDBS (dashed line with triangles); n=5. Error bars represent SEM; *p<0.05, **p<0.01, ***p<0.001, ns—non-significant.

FIGS. 12A-12D show that tonic nRE stimulation started during the prodromal disease stage prevents later impairments of spatial working memory in fAD mice. (A) Experimental design: spatial working memory (SWM) test via delta maze. GA: 3 h of 1.5% isoflurane. Ephys—LFP recording and IES analysis at age 4-5 months, after the first DBS. (B,C) Impairments of SWM in old (8-9-months APP/PS1mice: −DBS old) compared to young (4-5-months. AP/PS1mice: −DBS young) in the delta maze was prevented by tDBS-nRE (+DBS old). One-way ANOVA with Sidak's multiple comparison tests: 60 sec delay: −DBS old vs. +DBS old: P=0.024, −DBS old vs. −DBS young: P=0.002; n=7 per group old (5 males, 2 females) and n-8 young (males). 90 s delay: −DBS old vs. +DBS old: p=0.018, −DBS old vs. −DBS young: p=0.0013. (D) Frequency of CA1-IES during GA in APP/PS1mice at young age inversely correlates to spatial working memory performance at the 60 seconds delay (Spearman r=−0.65, p=0.0198) and 90 s delay (Spearman r=−0.76, p=0.0035) of the same mice at old age. Error bars represent SEM; ns—non-significant, *p<0.05, **p<0.01.

### DETAILED DESCRIPTION

In order to study the functional changes in hippocampal circuits before the onset of cognitive decline in mouse models for familial Alzheimer's disease (fAD), large-scale in vivo Ca²⁺ imaging and electrophysiology experiments were conducted. As demonstrated in the Experimental section herein, while neural dynamics and mean firing rates (MFRs) of hippocampal CA1 region neuronal populations were preserved in fAD mice during active wakefulness (Example 1), they were disrupted during non-rapid eye movement (NREM) sleep and anesthesia, resulting in pathological CA1 hyperexcitability (Examples 2, 4). This local dysregulation of CA1 MFRs was also shown to be related to an impairment of spatial working memory in fAD mice (Example 6), and to precede global disturbances in slow-wave activity (SWA) during NREM sleep (Example 3). Studying network-level firing rate homeostasis ex vivo suggests that fAD mutations disrupt the basic regulation of MFR set points by general anesthetic and homeostatic response to inhibition blockade, resulting in pathological MFR set points (Example 7). Teriflunomide (TERI), an inhibitor of the mitochondrial enzyme dihydroorotate dehydrogenase (DHODH), a recently identified signaling pathway of MFR set point down-regulation (Styr et al., 2019), was able to suppress CA1 hyperexcitability in anesthetized fAD mice, displaying unimpaired DHODH activity (Example 8). Overall, our study identifies a central role of low-arousal brain states in early vulnerability of hippocampal circuits in fAD models. Furthermore, it proposes that lowering firing rate set points during such states (e.g., NREM sleep) may present a new conceptual strategy for treating, preventing, or reducing likelihood of, pathological hippocampal activity during the pre-symptomatic AD phase.

As further shown, synaptic connections in the nucleus reuniens (nRE) of the midline thalamus, the principal source of thalamic inputs to the hippocampal CA1 region, are also disrupted by anesthesia in fAD mice (Example 6); and tonic deep brain stimulation (tDBS) of the nRE (tDBS-nRE), in sharp contrast to phasic DBS of the nRE (pDBS-nRE), significantly suppresses CA1 hyperexcitability (Examples 10-11), may thus be useful in restoring cognitive function, synaptic plasticity, and state-dependent neural activity patterns in fAD mice (Example 12), and when delivered during the prodromal disease stage, effectively prevent age-dependent memory impairments in APP/PS1 mice (Example 13).

Therefore, in accordance with the present invention, it has been surprisingly found that pre-symptomatic mice of several AD mouse models present with detectable epileptiform cortico-hippocampal hyperactivity during specific low-arousal brain states such as sleep and general anesthesia. This cortico-hippocampal hyperactivity includes diminished negative regulation of network MFR compared to wild-type mice, as well as abnormal high-voltage spikes in both the hippocampal CA1 and the medial prefrontal cortex (mPFC), which were not detected in wild-type mice. Such cortico-hippocampal hyperactivity has been previously detected mainly during sleep in symptomatic individuals. These findings mean that the cortico-hippocampal hyperactivity associated with low-arousal brain states precedes the clinical symptoms of the disease associated with them, and thus provides the basis for a non-invasive test for determining whether an asymptomatic individual is already in a process of developing AD or MCI.

As opposed to individuals simply at risk for AD or MCI because of genetic or other risk factors (see below), for whom it is unknown whether AD or MCI will present with clinical symptoms, and when, individuals who are already in a process of developing AD or MCI will be excellent candidates for treatment aimed at preventing (reducing likelihood of) or delaying the onset of clinical symptoms of AD or MCI.

The abnormal, or epileptiform, hyperactivity, or a metabolic correlate of the epileptiform hyperactivity, may be detected by various direct or indirect measurements of neuronal activity, including electroencephalogram (EEG), functional magnetic resonance imaging (fMRI), local field potential (LFP), positron emission tomography (PET) scan, and/or magnetoencephalography (MEG).

fMRI is a method for measuring brain activity by detecting changes associated with blood flow, relying on the fact that cerebral blood flow and neuronal activation are coupled. The primary form of fMRI uses the blood-oxygen-level dependent (BOLD) contrast imaging. This is a type of specialized brain and body scan used to map neural activity in the brain or spinal cord of humans or other animals by imaging the change in blood flow (hemodynamic response) related to energy use by brain cells. Accordingly, the changes in the blood-oxygen-level dependent contrast related to the hyperactivity can be detected by fMRI.

As further shown in the Experimental section below, the aforesaid abnormal cortico-hippocampal hyperactivity during sleep or anesthesia is also indicative of a risk for delirium or cognitive dysfunction resulting from general anesthesia, such as post-operative cognitive dysfunction (POCD) or post-operative delirium, and can therefore be used to determine whether a person about to undergo general anesthesia, e.g., as a part of a medical procedure, is at risk for developing post-operative delirium or cognitive dysfunction.

Finally, disclosed herein is a novel treatment based on DBS of the thalamic nRE, which is capable of reducing the cortico-hippocampal hyperactivity, and consequently preventing (reducing likelihood of) or attenuating delirium or cognitive dysfunction resulting from general anesthesia such as POCD or post-operative delirium. This treatment is therefore suitable for use, either for preventing (reducing likelihood of) disease progress and appearance of clinical symptoms in individuals identified as having cortico-hippocampal hyperactivity, or for preventing (reducing likelihood of) delirium and cognitive dysfunction in such individuals who are about to undergo general anesthesia. In some embodiments, the treatment can be administered after referring the subject of the treatment for Diagnostic Method A or Diagnostic Method B (as defined hereinbelow), which indicate that the subject is in the process of developing AD or MCI, or at risk for developing post-anesthesia delirium or cognitive dysfunction, respectively.

Moreover, the experimental data shown herein in fact demonstrate that the nRE of the midline thalamus is a key node in a neural circuit responsible for regulating resilience to AD. Various manipulations of nRE activity, including pharmacological inhibition of nRE spiking, chemogenetic inactivation of nRE-CA1 synapses, and tDBS-nRE, all suppressed CA1 hyperexcitability under anesthesia in fAD mice. Conversely, pDBS-nRE exacerbated CA1 hyperexcitability. The suppression of hyperexcitability by tDBS-nRE resulted in a reduction of CA1 and mPFC epileptiform discharges, along with a restoration of state-dependent regulation of CA1 firing rates. Most importantly, this tDBS-nRE intervention effectively prevented both the synaptic and cognitive aftermath of anesthesia, as well as age-dependent memory decline. Remarkably, these positive outcomes were observed even when tDBS-nRE was applied to awake, freely behaving mice.

Accordingly, in one aspect, the present invention provides a method of determining whether a subject is in a process of developing AD or MCI (also referred to herein as “Diagnostic Method A”), said method comprising detecting the presence of epileptiform spikes in the brain of said subject during a low-arousal brain state, wherein said subject is an asymptomatic subject, i.e., does not present clinical symptoms associated with said AD or MCI; and the presence of epileptiform spikes indicates that said subject is in the process of developing said AD or MCI.

Cortico-hippocampal hyperactivity refers to an increase in the neuronal excitability and synaptic transmission in the cortex and/or hippocampus, i.e., to cortical hyperactivity and/or hippocampal hyperactivity, reflected, e.g., by clinically silent seizures and epileptiform spikes (Lam et al., 2017; Vossel et al., 2013; Vossel et al., 2016).

As used herein, the term “epileptiform spikes” refers to high-voltage abnormal spikes, detectable at the CA1 in the hippocampus, not including spikes having known complicated and unique shapes and patterns which are associated with specific conditions other than AD and MCI.

The epileptiform aberrant or pathological high-voltage spikes, when detected by EEG recordings, are of a significantly higher voltage than the local average. At least based on experiments in mice, these spikes may be defined by setting a threshold of about 10 z-scores above and below the mean voltage during the suppression epochs of anesthesia, or about 8 z-scores above and below the mean if burst suppression ratio (BSR) is not analyzed. The high-voltage spikes are also characterized by a rapid rise and sink time, and have a maximal peak-to-peak duration of 30 milliseconds.

Accordingly, in some embodiments, the epileptiform spikes are of a significantly higher voltage compared to the local average spike voltage. In some embodiments, the epileptiform spikes are defined by setting a threshold of about 10 z-scores above and below the mean voltage during the suppression epochs of anesthesia. In some embodiments, the epileptiform spikes are defined by about 8 z-scores above and below the mean voltage if BSR is not analyzed. In some embodiments, the epileptiform spikes are defined by a maximal peak-to-peak duration of about 30 milliseconds, or of less than 30 milliseconds, e.g., up to about 25, about 20, about 15, about 10, or about 5, milliseconds.

The term “low-arousal brain state” as used herein refers to anesthesia, more particularly general anesthesia or diagnostic anesthesia, or sleep. In some embodiments, the low-arousal brain state is diagnostic anesthesia. In other embodiments, the low-arousal brain state is sleep, e.g., NREM sleep.

The term “general anesthesia” relates to a medically induced coma with loss of protective reflexes, resulting from the administration of one or more general anesthetic agents. General anesthesia of about 3 hours or more is associated with a risk of developing delirium or cognitive dysfunction as subsequent to the general anesthesia.

A subject may undergo general anesthesia for various reasons, which may be medical or non-medical reasons. Most commonly, general anesthesia is administered to enable an operative procedure (surgery), so a subject may undergo a medical procedure, such as surgery, under general anesthesia. However, other reasons for undergoing general anesthesia may include, e.g., dental treatments, electro-convulsive shock therapy, endotracheal intubation, and mechanical ventilation.

In some embodiments, the duration of the general anesthesia is about 3 hours; and in other embodiments, the duration of the general anesthesia is more than 3 hours, e.g., about 3.5 hours, 4 hours, 4.5 hours, 5 hours, 5.5 hours, 6 hours, 8 hours, 10 hours, or more. As used herein, the term “general anesthesia” encompasses both moderate and deep anesthesia. Moderate anesthesia (“conscious sedation”) is a drug-induced depression of consciousness during which patients respond purposefully following repeated or painful stimulation, and spontaneous ventilation is adequate. Deep anesthesia is a drug-induced depression of consciousness during which patients do not respond purposefully to repeated or painful stimulation, and spontaneous ventilation may be inadequate.

The term “diagnostic anesthesia”, used herein as opposed to “general anesthesia”, refers to a short anesthesia which is used for recording the EEG. As opposed to general anesthesia, which is long (about 3 hours or more) and involves a risk of post-anesthesia delirium or cognitive dysfunction such as post-operative cognitive dysfunction (POCD) or post-operative delirium, diagnostic anesthesia is substantially shorter, e.g., ≤1 hour, and does not involve a risk for post-anesthesia delirium or cognitive dysfunction.

In some embodiments, the duration of the diagnostic anesthesia is about 1.5 hour or less, such as up to about 80 minutes, up to about 70 minutes, up to about 60 minutes, up to about 50 minutes, up to about 40 minutes, up to about 30 minutes, up to about 20 minutes, or up to about 10 minutes. Such diagnostic anesthesia may be induced in a subject by administration of any known agent used in the practice, including but not limited to isoflurane, sevoflurane, ketamine-xylazine, medetomidine, or propofol.

The term “subject” as used herein refers to a mammal that is either a human (herein also referred to as “individual”) or a non-human animal such as horse, dog, cat, cow, and goat.

The terms “asymptomatic subject” or “asymptomatic individual” used herein interchangeably, refer to a subject/individual who does not present clinical symptoms associated with AD and/or MCI, as listed, e.g., in the diagnostic and statistical manual of mental disorders (DSM-5). This does not preclude the asymptomatic subject from presenting with symptoms that are not associated with AD or with MCI.

The term “clinical symptoms” as used herein relates to disturbing pathological phenotypes manifested by AD or MCI, such as memory loss, working memory problems, sleep problems, confusion, slowness, changes in mood, etc.

The phrase “in a process of developing AD or MCI”, as used herein with reference to a subject, relates to a subject who does not show any clinical symptoms, i.e., pathological phenotypes, associated with or related to AD or MCI, but is expected to show such symptoms within a certain amount of time, such as within several months, or within about 1, about 2, about 3, about 4, about 5, about 6, about 7, about 8, about 9, or about 10 to about 20, years, since AD or MCI has already started to develop in that subject.

In some embodiments, the subject tested according to Diagnostic Method A is about to undergo general anesthesia, e.g., a medical procedure such as a surgery under general anesthesia. As previously found, AD patients may be at increased risk of cognitive deterioration following anesthesia, i.e., general anesthesia is expected to accelerate the process of developing AD or MCI, when present. Accordingly, testing for the presence of cortico-hippocampal hyperactivity in asymptomatic individuals can identify individuals who are already in a process of developing AD or MCI, and are at risk for worsening or exacerbating said process as a result of the general anesthesia.

In some embodiments, the subject tested according to Diagnostic Method A is at risk for developing AD or MCI, such as a subject having a genetic predisposition to and/or a family history of AD or MCI, or a subject older than 60, or than 65, years old.

The phrase “subject at risk for developing AD or MCI” is defined herein as a subject having a predisposition to said diseases, that may be a genetic predisposition to said diseases, or a non-genetic predisposition involving other risk factors such as age, background diseases (vascular disease, heart disease, diabetes, stroke, etc.) or environmental factors (e.g., head injury). A genetic predisposition (or genetic susceptibility) is defined (based on the definition of the National Cancer Institute) as an increased likelihood or chance of developing a particular disease due to the presence of one or more gene mutations and/or a family history that indicates an increased risk of the disease. For example, a genetic predisposition to AD results from mutations in at least one of a number of genes, most commonly one of the APOE family genes, amyloid precursor protein (APP), and presenilin 1/2 (PSEN1/2).

In terms of age, it is known that individuals of an advanced age, such as 65 or above, have a higher tendency to present symptoms of such a disease. However, since the preclinical phase of, e.g., AD, is about 10-20 years, subjects who are otherwise already in a risk group for developing AD or MCI, may be tested according to the present invention already at the age of about 45 years old. Accordingly, in some embodiments, the subject at risk for developing AD or MCI, is at least 45, 50, 55, 60, 65, 70, 75, or 80, years old.

The method disclosed herein comprises detecting the presence of epileptiform spikes in the brain of the subject during a low-arousal brain state, e.g., diagnostic anesthesia. In some embodiments, the epileptiform spikes are detected at the hippocampus, such as at the CA1, or at the mPFC. According to the present invention, the detection of epileptiform spikes in the brain, more particularly at the hippocampus, such as at the CA1 or at the mPFC, may be performed using any suitable technique, e.g., by EEG, fMRI, PET scan, and/or MEG.

Once abnormal epileptiform high-voltage spikes have been detected in a subject as explained above, the subject is no longer merely at (a theoretical) risk for developing AD or MCI, but is already in a process of developing AD or MCI, and presenting with clinical symptoms is only a matter of time. Therefore, starting with treatment to prevent (reduce likelihood of) or delay the onset of said clinical symptoms would be beneficial.

Accordingly, in some embodiments, the subject tested according to Diagnostic Method A, as referred to in any one of the embodiments above, has been identified as presenting epileptiform spikes in the brain during diagnostic anesthesia, and said subject is subsequently provided with a treatment capable of attenuating or reducing cortico-hippocampal hyperactivity, thereby delaying the onset of symptoms of said AD or MCI. In other embodiments, the subject tested according to Diagnostic Method A, as referred to in any one of the embodiments above, has been identified as presenting epileptiform spikes in the brain during sleep or general anesthesia, and said subject is subsequently provided with a treatment capable of attenuating or reducing cortico-hippocampal hyperactivity, thereby delaying the onset of symptoms of said AD or MCI.

The term “treatment” or “treating” as used herein with respect to AD or MCI refers to administration of a treatment to either a subject presenting at least one pathological phenotype (clinical symptom) manifested by said AD or MCI, or an asymptomatic subject identified as being in a process of developing AD or MCI, in order to treat, reduce, attenuate, or ameliorate said pathological phenotype, when present, and/or slow down the progression of said AD or MCI, i.e., prevent or reduce likelihood of the appearance, or delay the onset, of pathological phenotypes if yet not present, or of additional pathological phenotypes, associated with said AD or MCI. Such a treatment may further result in improvement of memory functions and sleep quality.

The terms “preventing” and “delaying the onset” as used herein with respect to AD or MCI, and clinical symptoms thereof, refer to administration of the treatment to a subject, more particularly a subject identified as being in a process of developing AD or MCI, prior to the appearance or onset of pathological phenotypes (clinical symptoms) manifested by AD or MCI, in order to prevent or reduce the likelihood of the appearance or delay the onset of the clinical symptoms.

WO 2018096538 by the same applicant, herein incorporated by reference in its entirety, discloses treatment of diseases or disorders associated with cortico-hippocampal hyperactivity, such as epilepsy, AD, and MCI, by administration of an active agent capable of reducing DHODH enzyme activity in the central nervous system (CNS), thereby reducing the elevated cortico-hippocampal activity. Such a treatment may therefore also be suitable as a preventative treatment, similar to DBS, for individuals diagnosed by Diagnostic Method A as developing AD or MCI.

Accordingly, the phrase “a treatment capable of attenuating or reducing cortico-hippocampal hyperactivity” refers to certain treatments, demonstrated either in the Experimental section herein or in WO 2018096538 as being capable of reducing the cortico-hippocampal hyperactivity. Such treatments include administration of an active agent capable of reducing DHODH enzyme activity in the CNS, administration of an antiepileptic drug (AED), and application of DBS, preferably to the thalamic nRE.

In certain embodiments, the treatment capable of attenuating or reducing cortico-hippocampal hyperactivity comprises administration of an active agent capable of reducing DHODH enzyme activity in the CNS, wherein said active agent comprises a nucleic acid molecule capable of directly or indirectly reducing the gene expression level of DHODH enzyme.

siRNA molecules are short double stranded RNA molecules capable of reducing the expression level of a protein by inhibiting, reducing or eliminating gene expression through degradation of the target mRNA (in case of perfect match) or inhibition of mRNA translation (in case of imperfect match). The siRNA molecules may be artificial siRNA.

The term “shRNA” refers to an artificial double-stranded small hairpin RNA having a stem-loop structure and comprising 19-29 nucleotide. The shRNA is capable of reducing the expression level of a protein by inhibiting, reducing or eliminating gene expression through degradation of the target mRNA (in case of perfect match) or inhibition of mRNA translation (in case of imperfect match).

In certain particular such embodiments, said nucleic acid molecule capable of reducing the gene expression level of DHODH enzyme comprises an artificial and/or isolated siRNA or shRNA molecule comprising a nucleic acid sequence being complementary to a sequence within a nucleic acid sequence encoding said DHODH enzyme, or a nucleic acid molecule encoding said artificial siRNA or shRNA molecule. In some more particular such embodiments, the subject administered with said active agent (nucleic acid) is a human, and said DHODH enzyme is a human DHODH enzyme. In certain such embodiments, the isolated/artificial siRNA or shRNA molecule comprises a nucleic acid sequence having a sequence identity of 90% or more, e.g., about 95% or more, about 98% or more, or about 99%, identity to a sequence within said nucleic acid sequence encoding the DHODH enzyme; or a nucleic acid sequence being perfectly complementary to a sequence within said nucleic acid sequence encoding the DHODH enzyme.

In other particular such embodiments, the active agent capable of reducing DHODH enzyme activity in the CNS is a vector comprising said nucleic acid molecule. In more particular such embodiments, said vector is a modified virus derived from a virus selected from retrovirus, adenovirus, adeno-associated virus, pox virus, alphavirus, herpes virus, or lentivirus. Certain specific such embodiments are those wherein said vector is a modified virus derived from a lentivirus, i.e., a lentiviral-based shRNA delivery system. In particular such embodiments, said vector comprises a nucleic acid molecule encoding an shRNA molecule comprising a nucleic acid sequence being complementary to a sequence within a nucleic acid sequence encoding said DHODH enzyme.

In certain embodiments, the treatment capable of attenuating or reducing cortico-hippocampal hyperactivity comprises administration of an active agent capable of reducing DHODH enzyme activity in the CNS, wherein said active agent is a small molecule capable of reducing the activity of DHODH enzyme in the CNS, i.e., a DHODH inhibitor, or a pharmaceutically acceptable salt thereof. Examples of DHODH inhibitors include, without being limited to, 5-methyl-N-[4-(trifluoromethyl)phenyl]-isoxazole-4-carboxamide (leflunomide) or its metabolite (2Z)-2-cyano-3-hydroxy-N-[4-(trifluoromethyl)phenyl]but-2-enamide(teriflunomide); 6-fluoro-2-[4-(2-fluorophenyl)phenyl]-3-methylquinoline-4-carboxylic acid (brequinar); and 3-(3-chlorophenyl)-6,7-dihydro-5H-benzofuran-4-one (DD264). In particular embodiments the DHODH inhibitor is leflunomide or teriflunomide, preferably teriflunomide, or a pharmaceutically acceptable salt thereof.

Suitable pharmaceutically acceptable salts of the DHODH inhibitor include both acid addition salts and base addition salts of said DHODH inhibitor. Examples of acid addition salts include, without limiting, the mesylate salt, the maleate salt, the fumarate salt, the tartrate salt, the hydrochloride salt, the hydrobromide salt, the esylate salt, the p-toluenesulfonate salt, the benzenesulfonate salt, the benzoate salt, the acetate salt, the phosphate salt, the sulfate salt, the citrate salt, the carbonate salt, and the succinate salt. Non-limiting examples of base addition salts include metal salts such as alkali metal salts, e.g., lithium, sodium or potassium salts, and alkaline earth metal salts, e.g., calcium or magnesium salts; and salts of ammonium (NH₄⁺) or an organic cation derived from an amine of the formula R₄N⁺, wherein each one of the Rs independently is H, C₁-C₂₂, preferably C₁-C₆, alkyl, such as methyl, ethyl, propyl, isopropyl, n-butyl, sec-butyl, isobutyl, tert-butyl, n-pentyl, 2,2-dimethylpropyl, n-hexyl, and the like, phenyl, or heteroaryl such as pyridyl, imidazolyl, pyrimidinyl, and the like, or two of the Rs together with the nitrogen atom to which they are attached form a 3-7 membered ring optionally containing a further heteroatom selected from N, S and O, such as pyrrolydine, piperidine and morpholine.

Additional pharmaceutically acceptable salts of the DHODH inhibitor include salts of a cationic lipid or a mixture of cationic lipids. Cationic lipids are often mixed with neutral lipids prior to use as delivery agents. Neutral lipids include, but are not limited to, lecithins; phosphatidylethanolamine; diacyl phosphatidylethanolamines such as dioleoyl phosphatidylethanolamine, dipalmitoyl phosphatidylethanolamine, palmitoyloleoyl phosphatidylethanolamine and distearoyl phosphatidylethanolamine; phosphatidylcholine; diacyl phosphatidylcholines such as d dioleoyl phosphatidylcholine, dipalmitoyl phosphatidylcholine, palmitoyloleoyl phosphatidylcholine and distearoyl phosphatidylcholine; phosphatidylglycerol; diacyl phosphatidylglycerols such as dioleoyl phosphatidylglycerol, dipalmitoyl phosphatidylglycerol and distearoyl phosphatidylglycerol; phosphatidylserine; diacyl phosphatidylserines such as dioleoyl- or dipalmitoyl phosphatidylserine; and diphosphatidylglycerols; fatty acid esters; glycerol esters; sphingolipids; cardiolipin; cerebrosides; ceramides; and mixtures thereof. Neutral lipids also include cholesterol and other 3β hydroxy-sterols.

Examples of cationic lipid compounds include, without limiting, Lipofectin® (Life Technologies, Burlington, Ontario) (1:1 (w/w) formulation of the cationic lipid N-[1-(2,3-dioleyloxy)propyl]-N,N,N-trimethylammonium chloride and dioleoylphosphatidyl-ethanolamine); Lipofectamine™ (Life Technologies, Burlington, Ontario) (3:1 (w/w) formulation of polycationic lipid 2,3-dioleyloxy-N-[2(spermine-carboxamido)ethyl]-N,N-dimethyl-1-propanamin-iumtrifluoroacetate and dioleoylphosphatidyl-ethanolamine), Lipofectamine Plus (Life Technologies, Burlington, Ontario) (Lipofectamine and Plus reagent), Lipofectamine 2000 (Life Technologies, Burlington, Ontario) (Cationic lipid), Effectene (Qiagen, Mississauga, Ontario) (Non liposomal lipid formulation), Metafectene (Biontex, Munich, Germany) (Polycationic lipid), Eu-fectins (Promega Biosciences, San Luis Obispo, Calif.) (ethanolic cationic lipids numbers 1 through 12: C₅₂H₁₀₆N₆O₄.4CF₃CO₂H, C₈₈H₁₇₈N₈O₄S₂.4CF₃CO₂H, C₄₀H₈₄NO₃P.CF₃CO₂H, C₅₀H₁₀₃N₇O₃.4CF₃CO₂H, C₅₅H₁₁₆N₈O₂.6CF₃CO₂H, C₄₉H₁₀₂N₆O₃.4CF₃CO₂H, C₄₄H₈₉N₅O₃.2CF₃CO₂H, C₁₀₀H₂₀₆N₁₂O₄S₂.8CF₃CO₂H, C₁₆₂H₃₃₀N₂₂O₉.13CF₃CO₂H, C₄₃H₈₈N₄O₂.2CF₃CO₂H, C₄₃H₈₈N₄O₃.2CF₄CO₂H, C₄₁H₇₈NO₈P); Cytofectene (Bio-Rad, Hercules, Calif.) (mixture of a cationic lipid and a neutral lipid), GenePORTER® (Gene Therapy Systems, San Diego, Calif.) (formulation of a neutral lipid (Dope) and a cationic lipid) and FuGENE 6 (Roche Molecular Biochemicals, Indianapolis, Ind.) (Multi-component lipid based non-liposomal reagent).

Pharmaceutically acceptable salts of the DHODH inhibitor may be formed by conventional means, e.g., by reacting a free base form of the DHODH inhibitor with one or more equivalents of the appropriate acid in a solvent or medium in which the salt is insoluble, or in a solvent such as water which is removed in vacuo or by freeze drying, or by exchanging the anion/cation of an existing salt for another anion/cation on a suitable ion exchange resin.

In certain embodiments, the treatment capable of attenuating or reducing cortico-hippocampal hyperactivity comprises administration of an AED. Examples of AEDs include, without being limited to, levetiracetam, brivaracetam, ethosuximide, and stiripentol.

The administration of an active agent capable of reducing DHODH enzyme activity in the CNS, as defined in any one of the embodiments above, or an AED, may be performed by any suitable administration route, e.g., intravenously, intraarterially, intrathecally, intracerebroventricularly, intrapleurally, intratracheally, intraperitoneally, intramuscularly, subcutaneously, topically, orally, or by inhalation. In some embodiments, for example when said active agent or AED is not capable of crossing, i.e., penetrating, the blood-brain-barrier (such as when the active agent is teriflunomide), administration should be directed into the brain or the spinal cord, such as intrathecal administration, or intracerebroventricular administration, which bypasses the blood-brain barrier and other mechanisms that limit drug distribution into the brain, allowing high drug concentration to enter the CNS. In other embodiments, for example when the active agent or AED is capable of penetrating the blood-brain barrier, administration may be performed either orally or parenterally.

In certain embodiments, the treatment capable of attenuating or reducing cortico-hippocampal hyperactivity comprises application of DBS, preferably to the thalamic nRE.

In particular such embodiments, the DBS protocol used as treatment according to the present invention is a tonic DBS protocol, i.e., a DBS protocol involving stimuli continuously delivered at a constant frequency (as opposed to a phasic DBS protocol, which involves bursts of stimuli with an inter-burst intervals), e.g., a low frequency tonic DBS protocol. In some embodiments, the frequency of the tonic DBS is lower than about 250 Hz, and in other embodiments, the frequency of the tonic DBS is lower than about 130 Hz, lower than about 100 Hz, lower than about 70 Hz, lower than about 50 Hz, lower than about 30 Hz, lower than about 25 Hz, lower than about 10 Hz, or lower than about 8 Hz. In some embodiments, the tonic DBS protocol involves a stimulus of up to about 250 Hz, and in other embodiments, the tonic DBS protocol involves a stimulus of about 2 to about 250 Hz, e.g., about 5 to about 200 or 150 Hz, about 10 to about 100 Hz, about 15 to about 50 Hz, about 20 to about 30 Hz, or about 25 Hz.

In some embodiments, the tonic DBS protocol used as treatment according to the present invention comprises pulses having a pulse duration of up to about 0.1, 0.2, 0.3, 0.4, 0.5, 0.6, 0.7, 0.8, 0.9, or 1.0 milliseconds. In other embodiments, said tonic DBS protocol comprises pulses having a pulse duration of about 0.01 to about 1.0 milliseconds, about 0.02 to about 0.8 milliseconds, about 0.03 to about 0.6 milliseconds, about 0.05 to about 0.5 milliseconds, about 0.1 to about 0.5 milliseconds, about 0.2 to about 0.5 milliseconds, about 0.3 to about 0.5 milliseconds, about 0.4 to about 0.5 milliseconds, or about 0.5 milliseconds. In further embodiments, said tonic DBS protocol comprises pulses having a pulse duration of about 0.01 to about 0.09 milliseconds, about 0.02 to about 0.08 milliseconds, about 0.03 to about 0.07 milliseconds, about 0.04 to about 0.06 milliseconds, or about 0.05 milliseconds.

In some embodiments, the tonic DBS protocol used as treatment according to the present invention involves pulses having a voltage of up to about 10.5 V, and in other embodiments, the tonic DBS protocol involves pulses having a voltage of lower than 10.5 V, e.g., pulses of about 8 V, about 5 V, about 4 V, about 3 V, about 2 V, about 1 V or less, about 0.1 to about 10.5 V, about 0.1 to about 8 V, about 0.1 to about 5 V, about 0.1 V to about 1 V, about 0.5 to about 8 V, about 0.5 to about 5 V, about 0.5 to about 4 V, about 0.5 to about 3 V, about 0.5 to about 2 V, or about 0.5 to about 1 V.

In some embodiments, the tonic DBS protocol used as treatment according to the present invention involves pulses having a current of up to about 10.5 mA, and in other embodiments, the tonic DBS protocol involves pulses having a current of lower than 10.5 mA, e.g., pulses of about 8 mA, about 5 mA, about 4 mA, about 3 mA, about 2 mA, about 1 mA or less, about 0.1 to about 10.5 mA, about 0.1 to about 8 mA, about 0.1 to about 5 mA, about 0.1 mA to about 1 mA, about 0.5 to about 8 mA, about 0.5 to about 5 mA, about 0.5 to about 4 mA, about 0.5 to about 3 mA, about 0.5 to about 2 mA, or about 0.5 to about 1 mA.

It is however noted that the voltage (V) and the current (I) are tied by the formula V=IR, when R represents the resistance of the system. Accordingly, depending on the resistance, when a certain voltage is selected, the current is defined by the formula I=V/R. Alternatively, when a certain current is selected, the voltage is defined by the formula V=IR.

The pulses are generally delivered during a relatively short period of time after electrode implantation and stimulus parameters optimization. In some embodiments, the pulses are delivered during a session length of up to 800, 1200, or 1600 seconds, and in other embodiments, the pulses are delivered during a session length of about 100 to about 1600 seconds, about 200 to about 1500 seconds, about 300 to about 1400 seconds, about 400 to about 1300 seconds, about 500 to about 1200 seconds, about 600 to about 1100, about 700 to about 1000 seconds, or about 800 to about 900 seconds. In some embodiments, the pulses are delivered in a single session. In some embodiments, the pulses are delivered in repeated, multiple sessions. In some embodiments, the pulses are delivered in repeated sessions at a session frequency of once a week, once every two weeks, once every three weeks, or once a month. In some embodiments, the pulses are delivered repetitively, with a session frequency of less than once a month, e.g., once every two months, once every three months, once every six months, or less than once every six months.

In some embodiments, the tonic DBS protocol used as treatment according to the present invention includes up to 20,000, 25,000, 30,000, 35,000, or 40,000 pulses per session, and in other embodiments, said tonic DBS protocol includes between about 4,000 and about 40,000 pulses per session, about 6,000 to about 38,000 pulse per session, about 8,000 to about 36,000 pulse per session, about 10,000 to about 34,000 pulse per session, about 12,000 to about 32,000 pulse per session, about 14,000 to about 30,000 pulse per session, about 16,000 to about 28,000 pulse per session, about 18,000 to about 26,000 pulse per session, or about 20,000 to about 24,000 pulse per session.

It is noted that the frequency (F), the session length (L), and the number of pulses per session (N) are tied by the formula F=N/L. Accordingly, for any two parameters of F, L, and N that are selected, the third is defined by this formula.

In some embodiments, the treatment according to the invention is a low frequency tonic DBS, i.e., a tonic DBS protocol involving stimuli continuously delivered at a constant frequency of up to about 250 Hz, e.g., of about 2 to about 250 Hz, about 5 to about 200 or 150 Hz, about 10 to about 100 Hz, about 15 to about 50 Hz, about 20 to about 30 Hz, or about 25 Hz, having a pulse duration, voltage, current, session length, and number of pulses per session, each as defined above considering the limitations defined above for parameters which are tied by a formula, wherein each combination of said pulse duration, voltage, current, session length, and number of pulses per session represents a separate embodiment. In each one of the embodiments defined herein, the treatment may be administered either in a single session, or in multiple sessions at any session frequency as defined above.

In some specific embodiments, a tonic DBS protocol used as a treatment according to the present invention includes about 10000 to about 15000 pulses, each of about 0.2 to about 5 mA and having a pulse duration of about 0.5 milliseconds, continuously delivered at a frequency of about 25 Hz during about 400-600 seconds. In some embodiments, a tonic DBS protocol suitable for the present invention includes about 10,000 pulses, each of about 0.5 to about 5 mA and having a pulse duration of about 0.5 milliseconds, continuously delivered at a frequency of about 25 Hz during about 400 seconds. In some embodiments, a tonic DBS protocol suitable for the present invention includes about 15000 pulses, each of about 0.5 to about 5 mA and having a pulse duration of about 0.5 milliseconds, continuously delivered at a frequency of about 25 Hz during about 600 seconds. In some embodiments, a tonic DBS protocol suitable for the present invention includes about 11000, 12000, 13000, or 14000 pulses, each of about 0.5 to about 5 mA and having a pulse duration of about 0.5 milliseconds, continuously delivered at a frequency of about 25 Hz during about 440-560 seconds.

In some specific embodiments, a tonic DBS protocol used as treatment according to the present invention includes about 10000 to about 15000 pulses, each of about 0.5 to about 4 V and having a pulse duration of about 0.5 milliseconds, continuously delivered at a frequency of about 25 Hz during about 400-600 seconds. In some embodiments, a tonic DBS protocol suitable for the present invention includes about 10000 pulses, each of about 0.5 to about 4 V and having a pulse duration of about 0.5 milliseconds, continuously delivered at a frequency of about 25 Hz during about 400 seconds. In some embodiments, a tonic DBS protocol suitable for the present invention includes about 15000 pulses, each of about 0.5 to about 4 V and having a pulse duration of about 0.5 milliseconds, continuously delivered at a frequency of about 25 Hz during about 600 seconds. In some embodiments, a tonic DBS protocol suitable for the present invention includes about 11000, 12000, 13000, or 14000 pulses, each of about 0.5 to about 4 V and having a pulse duration of about 0.5 milliseconds, continuously delivered at a frequency of about 25 Hz during about 440-560 seconds.

In some embodiments, the tonic DBS protocol used as treatment according to the present invention is a tonic DBS protocol according to any one of the embodiments above, which is applied to the thalamic nRE. Particular such embodiments are those wherein said tonic DBS is a low-frequency tonic DBS, i.e., a tonic DBS protocol involving stimuli continuously delivered at a constant frequency of up to about 250 Hz, e.g., of about 2 to about 250 Hz, about 5 to about 200 or 150 Hz, about 10 to about 100 Hz, about 15 to about 50 Hz, about 20 to about 30 Hz, or about 25 Hz.

According to the present invention, any treatment capable of attenuating or reducing cortico-hippocampal hyperactivity as defined herein, provided according to any one of the methods disclosed herein to a subject (regardless of whether suffering from AD or MCI, or identified as being in a process of developing AD or MCI), may be administered in parallel, i.e., in addition and without connection, to other treatments possibly given to said subject so as to treat other medical indications. For example, application of DBS, in particular tonic DBS and preferably to the thalamic nRE, may be performed in parallel to a different DBS-based treatment provided to said subject so as to treat, e.g., Parkinson disease.

In another aspect, the present invention provides a method of determining whether a subject about to undergo general anesthesia is at risk for developing post-anesthesia delirium or cognitive dysfunction (also referred to herein as “Diagnostic Method B”), said method comprising detecting epileptiform spikes in the brain of said subject during a low-arousal brain state, wherein said subject does not present clinical symptoms associated with AD or MCI; and the presence of epileptiform spikes in the brain of said subject indicates that said subject is in a process of developing said AD or MCI and thus at risk for developing post-anesthesia delirium or cognitive dysfunction.

In certain embodiments, the subject tested by Diagnostic Method B is about to undergo a medical procedure such as a surgery, under general anesthesia, and the post-anesthesia delirium or cognitive dysfunction is post-operative delirium or POCD.

The phrase “post-anesthesia delirium or cognitive dysfunction” as used herein encompasses conditions of delirium or cognitive dysfunction which develop in a subject following general anesthesia. The symptoms for these conditions most commonly include decline in memory, learning and intellectual abilities, as well as other symptoms detailed below with reference to POCD. POCD and post-operative delirium are the more common and well-known conditions encompassed by the definition of post-anesthesia delirium or cognitive dysfunction, which relate to general anesthesia administered to enable conducting a surgery. According to the Sunnybrook research institute, POCD refers to a state in which a patient's memory and learning decline after surgery. The most common symptoms include memory decline, inability to complete tasks that were easier before the surgery, decline in intellectual abilities, difficulty in multitasking, reduced psychomotor skills, problems with language comprehension and with social integration. The symptoms can manifest in varying degrees of severity. POCD may occur in about 1 of 3 patients, and may last up to 3 months after surgery in about 10% of the patients. POCD is usually associated with general anesthesia, typically of a relatively long duration, such as about 3 hours or more. Some of the risk factors include an age of at least 60 years, as well as pre-existing conditions including cognitive impairment and psychiatric disorders. Some studies have shown that patients who developed POCD may be more likely to develop long term cognitive impairments in the future, indicating that POCD may occur in patients with already “sensitive” brains that are at risk for these types of disorders.

Post-operative delirium is also rather common and involves patients having moments of disorientation, hallucinations and confusion interspaced with periods of being oriented and aware. This typically occurs immediately after surgery, for 24-72 hours postoperatively.

Since it was previously found that general anesthesia may cause post-anesthesia delirium or cognitive dysfunction such as POCD or post-operative delirium, it would be beneficial to find out whether a subject about to undergo general anesthesia has an increased risk for such post-anesthesia phenomena. As shown herein, cortico-hippocampal hyperactivity, found only in the pre-symptomatic AD mouse model and not in wild-type mice, was predictive of a working memory impairment following general anesthesia. Accordingly, testing an asymptomatic individual for the presence of cortico-hippocampal hyperactivity, prior to undergoing a procedure under general anesthesia, would provide important information prior to the procedure.

In some embodiments, the subject about to undergo general anesthesia is a subject at risk for developing AD or MCI as referred to above, or a subject who is at least 45, 50, 55, 60, 65, 70, 75, or 80, years old, e.g., older than 60 years old.

In some embodiments, Diagnostic Method B comprises detecting the presence of epileptiform spikes at the hippocampus, such as at the CA1, or at the mPFC, e.g., using EEG, fMRI, PET scan, and/or MEG. Said detection is preferably carried out during diagnostic anesthesia, but may alternatively be carried out during sleep.

In certain embodiments, the subject tested according to Diagnostic Method B, as referred to in any one of the embodiments above, has been identified as presenting with epileptiform spikes in the brain, e.g., during diagnostic anesthesia, and said subject is subsequently provided, prior to said general anesthesia, with a treatment capable of attenuating or reducing cortico-hippocampal hyperactivity as described above, thereby preventing (reducing likelihood of) or attenuating said post-anesthesia delirium or cognitive dysfunction.

In some embodiments, the treatment capable of attenuating or reducing cortico-hippocampal hyperactivity is selected from administration of an active agent capable of reducing DHODH enzyme activity in the CNS, such as a nucleic acid molecule, e.g., an siRNA molecule or an shRNA molecule, or a small molecule such as leflunomide, teriflunomide, brequinar, or DD264; administration of an AED such as levetiracetam, brivaracetam, ethosuximide, or stiripentol; and/or application of DBS, preferably to the thalamic nRE, and particularly a tonic DBS protocol according to any one of the embodiments above. More particular such protocols are low frequency tonic DBS protocols, i.e., tonic DBS protocols involving stimuli continuously delivered at a constant frequency of up to about 250 Hz, e.g., of about 2 to about 250 Hz, about 5 to about 200 or 150 Hz, about 10 to about 100 Hz, about 15 to about 50 Hz, about 20 to about 30 Hz, or about 25 Hz.

In yet another aspect, the present invention provides a method for preventing or delaying the onset of clinical symptoms of AD or MCI in a subject in need thereof (also referred to herein as “AD/MCI Preventing Method”), wherein said subject does not present clinical symptoms associated with said AD or MCI, and has been identified as presenting epileptiform spikes in the brain during a low-arousal brain state, indicating that said subject is in a process of developing said AD or MCI; and said method comprises providing said subject with a treatment capable of attenuating or reducing cortico-hippocampal hyperactivity, thereby preventing (reducing likelihood of) or delaying the onset of clinical symptoms associated with said AD or MCI.

In some embodiments, the subject treated according to the AD/MCI Preventing Method disclosed herein has been identified as presenting epileptiform spikes at the hippocampus, such as at the CA1, or at the mPFC, during anesthesia, e.g., using EEG, fMRI, PET scan, and/or MEG. Said detection is preferably carried out during diagnostic anesthesia, but may alternatively be carried out during sleep or general anesthesia.

In some embodiments, the subject treated according to the AD/MCI Preventing Method disclosed herein is about to undergo general anesthesia, e.g., a medical procedure under general anesthesia, which is expected to accelerate said process of developing said AD or MCI when present.

In some embodiments, the treatment capable of attenuating or reducing cortico-hippocampal hyperactivity, provided according to the AD/MCI Preventing Method disclosed herein according as referred to in any one of the embodiments above, is selected from administration of an active agent capable of reducing DHODH enzyme activity in the CNS, such as a nucleic acid molecule, e.g., an siRNA molecule or an shRNA molecule, or a small molecule such as leflunomide, teriflunomide, brequinar, or DD264; administration of an AED such as levetiracetam, brivaracetam, ethosuximide, or stiripentol; and application of DBS, preferably to the thalamic nRE, and particularly a tonic DBS protocol according to any one of the embodiments above. More particular such protocols are low frequency tonic DBS protocols involving stimuli continuously delivered at a constant frequency of up to about 250 Hz, e.g., of about 2 to about 250 Hz, about 5 to about 200 or 150 Hz, about 10 to about 100 Hz, about 15 to about 50 Hz, about 20 to about 30 Hz, or about 25 Hz. In particular specific embodiments, said treatment comprises administration of teriflunomide or a pharmaceutically acceptable salt thereof. In other particular embodiments, said treatment comprises DBS, more particularly a tonic DBS, and preferably low frequency tonic DBS, protocol applied to the thalamic nRE.

In still another aspect, the present invention provides a method for preventing (reducing likelihood of) or attenuating post-anesthesia delirium or cognitive dysfunction in a subject about to undergo general anesthesia, e.g., a medical procedure under general anesthesia (also referred to herein as “Post-Anesthesia Delirium Preventing Method”), wherein said subject does not present clinical symptoms associated with AD or MCI, and has been identified as presenting epileptiform spikes in the brain during a low-arousal brain state, indicating that said subject is in a process of developing AD or MCI; and said method comprises providing said subject, prior to said general anesthesia, with a treatment capable of attenuating or reducing cortico-hippocampal hyperactivity, thereby preventing (reducing likelihood of) or attenuating said post-anesthesia delirium or cognitive dysfunction.

In some embodiments, the subject treated according to the Post-Anesthesia Delirium Preventing Method disclosed herein has been identified as presenting epileptiform spikes at the hippocampus, such as at the CA1, or at the mPFC, during anesthesia, e.g., using EEG, fMRI, PET scan, and/or MEG. Said detection is preferably carried out during diagnostic anesthesia, but may alternatively be carried out during sleep or general anesthesia.

In some embodiments, the treatment capable of attenuating or reducing cortico-hippocampal hyperactivity, provided according to the Post-Anesthesia Delirium Preventing Method disclosed herein as referred to in any one of the embodiments above, is selected from administration of an active agent capable of reducing DHODH enzyme activity in the CNS, such as a nucleic acid molecule, e.g. an siRNA molecule or an shRNA molecule, or a small molecule such as leflunomide, teriflunomide, brequinar, or DD264; administration of an AED such as levetiracetam, brivaracetam, ethosuximide, or stiripentol, and application of DBS, preferably to the thalamic nRE, and particularly a tonic DBS protocol according to any one of the embodiments above. More particular such protocols are low frequency tonic DBS protocols involving stimuli continuously delivered at a constant frequency of up to about 250 Hz, e.g., of about 2 to about 250 Hz, about 5 to about 200 or 150 Hz, about 10 to about 100 Hz, about 15 to about 50 Hz, about 20 to about 30 Hz, or about 25 Hz. In particular specific embodiments, said treatment comprises administration of teriflunomide or a pharmaceutically acceptable salt thereof. In other particular embodiments, said treatment comprises DBS, more particularly a tonic DBS, and preferably low frequency tonic DBS, protocol applied to the thalamic nRE.

In a further aspect, the present invention provides a method of treating AD or MCI in a subject suffering from AD or MCI, i.e., a subject presenting with symptoms associated with AD or with MCI, or preventing (reducing likelihood of) or delaying the onset of clinical symptoms associated with said AD or MCI in an asymptomatic subject being in the process of developing AD or MCI, e.g., a subject that has been identified as presenting epileptiform spikes in the brain during a low-arousal brain state, said method also referred to herein as “Treating Method” and comprising applying DBS to the thalamic nRE of the brain of said subject, more particularly a tonic DBS protocol according to any one of the embodiments above. More particular such protocols are low frequency tonic DBS protocols involving stimuli continuously delivered at a constant frequency of up to about 250 Hz, e.g., of about 2 to about 250 Hz, about 5 to about 200 or 150 Hz, about 10 to about 100 Hz, about 15 to about 50 Hz, about 20 to about 30 Hz, or about 25 Hz.

In some embodiments, the asymptomatic subject treated according to the Treating Method disclosed herein has been identified as presenting epileptiform spikes at the hippocampus, such as at the CA1, or at the mPFC, during anesthesia, e.g., by using EEG, fMRI, PET scan, and/or MEG. Said detection is preferably carried out during diagnostic anesthesia, but may alternatively be carried out during sleep or general anesthesia.

In yet a further aspect, the present invention provides a method of treating post-anesthesia delirium or cognitive dysfunction in a subject in need thereof having undergone general anesthesia (also referred to herein as “Post-Anesthesia Delirium Treating Method”), said method comprising providing to said subject a treatment capable of attenuating or reducing cortico-hippocampal hyperactivity, selected from administration of an active agent capable of reducing DHODH enzyme activity in the CNS, such as a nucleic acid molecule, e.g., an siRNA molecule or an shRNA molecule, or a small molecule such as leflunomide, teriflunomide, brequinar, or DD264; administration of an AED such as levetiracetam, brivaracetam, ethosuximide, or stiripentol; and application of DBS, preferably to the thalamic nRE, more particularly a tonic DBS protocol according to any one of the embodiments above. More particular such protocols are low frequency tonic DBS protocols involving stimuli continuously delivered at a constant frequency of up to about 250 Hz, e.g., of about 2 to about 250 Hz, about 5 to about 200 or 150 Hz, about 10 to about 100 Hz, about 15 to about 50 Hz, about 20 to about 30 Hz, or about 25 Hz. In particular specific embodiments, said treatment comprises administration of teriflunomide or a pharmaceutically acceptable salt thereof. In other particular embodiments, said treatment comprises DBS, more particularly a tonic DBS, and preferably low frequency tonic DBS, protocol applied to the thalamic nRE.

In still a further aspect, the present invention provides an active agent capable of reducing DHODH enzyme activity in the CNS, or an AED, for use in preventing (reducing likelihood of) or delaying the onset of clinical symptoms associated with AD or MCI in a subject, wherein said subject does not present clinical symptoms associated with said AD or MCI, and has been identified as presenting epileptiform spikes in the brain during a low-arousal brain state, indicating that said subject is in a process of developing said AD or MCI.

In some embodiments, the subject provided with said active agent or AED has been identified as presenting epileptiform spikes at the hippocampus, such as at the CA1, or at the mPFC, during anesthesia, e.g., using EEG, fMRI, PET scan, and/or MEG. Said detection is preferably carried out during diagnostic anesthesia, but may alternatively be carried out during sleep or general anesthesia.

It some embodiments, the term “an active agent capable of reducing DHODH enzyme activity in the CNS, or an AED”, used herein, refer to a single such active agent or AED, each as defined above; and in other embodiments, said term refers to a combination of more than one such active agents and/or AEDs, administered concomitantly, or sequentially at any order.

In some embodiments, the subject provided with said active agent is about to undergo general anesthesia, e.g., a medical procedure such as a surgery under general anesthesia, which is expected to accelerate said process of developing said AD or MCI.

In some embodiments, said active agent capable of reducing DHODH enzyme activity in the CNS is a nucleic acid molecule, e.g., an siRNA molecule or an shRNA molecule, or a small molecule such as leflunomide, teriflunomide, brequinar, or DD264; and/or said AED is selected from levetiracetam, brivaracetam, ethosuximide, and stiripentol.

In yet a further aspect, the present invention provides an active agent capable of reducing DHODH enzyme activity in the CNS, or an AED, for use in preventing (reducing likelihood of) or attenuating post-anesthesia delirium or cognitive dysfunction, e.g., POCD or post-operative delirium, in a subject about to undergo general anesthesia, wherein said subject does not present clinical symptoms associated with AD or MCI, and has been identified as presenting epileptiform spikes in the brain during a low-arousal brain state, indicating that said subject is in a process of developing said AD or MCI.

In some embodiments, the subject provided with said active agent or AED has been identified as presenting epileptiform spikes at the hippocampus, such as at the CA1, or at the mPFC, during anesthesia, e.g., using EEG, fMRI, PET scan, and/or MEG. Said detection is preferably carried out during diagnostic anesthesia, but may alternatively be carried out during sleep or general anesthesia.

In some embodiments, the active agent capable of reducing DHODH enzyme activity in the CNS is a nucleic acid molecule, e.g., an siRNA molecule or an shRNA molecule, or a small molecule such as leflunomide, teriflunomide, brequinar, or DD264; and/or said AED is selected from levetiracetam, brivaracetam, ethosuximide, or stiripentol.

For purposes of clarity, and in no way limiting the scope of the teachings, unless otherwise indicated, all numbers expressing quantities, percentages or proportions, and other numerical values recited herein, should be interpreted as being preceded in all instances by the term “about”, regardless of whether “about” is explicitly prepended to the numerical value. Accordingly, unless indicated to the contrary, the numerical parameters set forth in this specification are approximations that may vary by up to plus or minus 10% depending upon the desired properties to be obtained by the present invention.

The invention will now be illustrated by the following non-limiting examples.

### EXAMPLES

### Materials and Methods

Mice. All animal experiments were approved by the Tel Aviv University Committee on Animal Care. In vivo Ca²⁺ imaging experiments were performed on 4-6-month-old male and female APP/PS1 (APP_{Swe}/PS1ΔE9) hemizygotes (Stock No. 005864, The Jackson Laboratory) and their wild-type littermates. All mice were on a C57BL/6J-congenic background. All animals were kept in a normal light/dark cycle (12 h/12 h, lights on at 7 AM). All animals except for mice undergoing delta maze experiments had free access to food and water. Mutant and wild-type mice were housed together for behavioral, biochemical and electroencephalogram (EEG)/electromyography (EMG) experiments. Mice for electrophysiology and Ca²⁺ imaging were singly housed after implantation.

General surgical procedures. In all the surgical procedures, the mice were anaesthetized with 5% isoflurane by volume for induction, injected i.p. with ketamine/xylazine (100 mg/kg ketamine and 8 mg/kg xylazine), head fixed to a stereotaxic apparatus (David Kopf instruments) and then maintained anesthetized by continuous isoflurane (1.5%) inhalation throughout the surgical procedure. Eye ointment was used to protect the mice eyes (Duratears, Vetmarket), and body temperature was recorded and maintained by a heating pad (FHC, DC temperature controller) at 34° C. throughout the surgery. At the beginning of each surgical procedure the mice were injected subcutaneous with Carprofen (5 mg/kg) to reduce inflammation and pain. The mice were then allowed to recover in their home cage for at least 1-2 weeks before the subsequent surgical procedure or experiment began. When studying the effects of distinct anesthetics on physiological CA1 properties, individual anesthetics were used as specified in the figure legends.

For intracerebroventricular (ICV) ainjections, a small hole was drilled in the skull above the left lateral ventricle (0.7 mm posterior, 1.2 mm lateral to bregma), and a 5 mm guide cannula was slowly inserted into the ventricle and fixed to the skull by dental cement (C&B Metabond, Parkell). The guide cannula was sealed with a 5 mm sterile metal bar to prevent CSF leakage and possible infections. 1-2 weeks after the surgery, the mice received ICV injections using a 10 μl syringe (Hamilton). The mice were injected with 1 μl containing 27 μg of teriflunomide (TERI), dissolved in DMSO or vehicle (1 μl of DMSO) with speed of 0.15 μl/min (Nano Jet stereotaxic syringe pump).

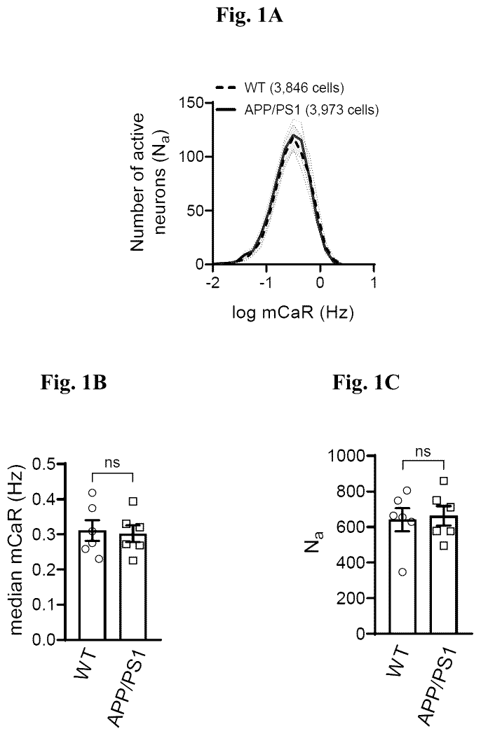

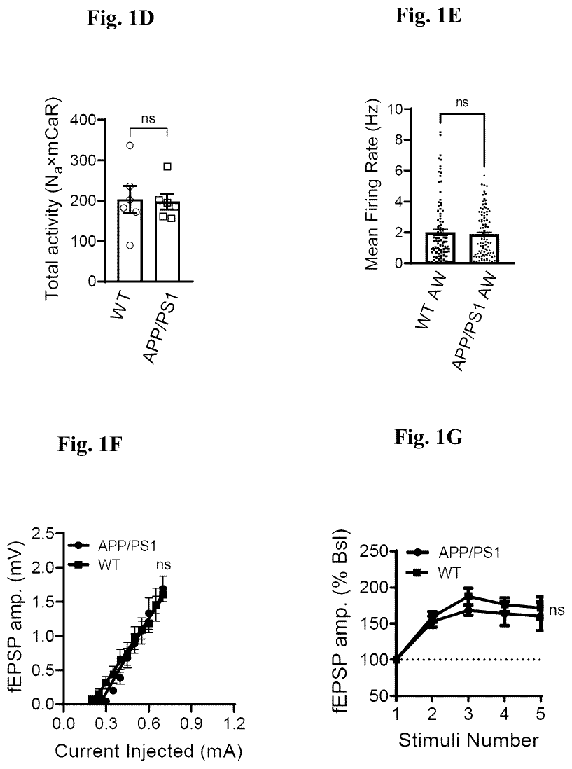

LFP/fEPSP recordings. Small diameter holes were drilled in the skull at the position of the recording and stimulating electrodes. The recording electrode (bipolar, PFA-coated stainless steel; 0.071-0.127 mm diameter) was slowly lowered through the cortex into the CA1 (2.06 mm posterior to bregma; 1.5 mm mediolateral, ML; 1.5 mm dorsoventral from bregma, DV), and the stimulating electrode (bipolar stainless steel; 0.071-0.127 mm diameter) was slowly lowered through the cortex into the Schaffer Collateral (2.54 mm posterior to bregma; 2.75 mm ML; 2.2 mm DV). Ground electrode was screwed to the skull above the cerebellum. Test stimuli of 50-100 μA were delivered to the SC at 0.06 Hz to verify the proper location of the electrodes and to estimate the stability of the signal. The electrodes were firmly fixed to the skull by dental cement (C&B Metabond, Parkell) and dental acrylic. Operated mice recovered in their home cage for at least a week following electrodes implantation. For evoked field excitatory postsynaptic potential (fEPSP) recordings in CA3-CA1 synapses in awake mice (FIGS. 1F-1G), mice were habituated for a few days to the experimental device that was composed from a running wheel and metal bar in which the mice were head fixed into throughout the experiment with a screw. Following habituation, the mice were head fixed to the experimental device, and CA3-CA1 fEPSP measurements were taken 30 minutes after.

Extracellular field potentials and electromyograms were amplified ×100 using a custom-made amplifier, band-pass filtered between 0.1 Hz and 4 KHz, and digitized by Digidata 1440A at 56 kHz sampling rate (Molecular Devices). Data were analysed using Clampfit 10.7 (Molecular devices) for fEPSP's or custom MATLAB functions (MathWorks).

EEG/EMG recordings. EEG screws were placed over the frontal and parietal cortices. Ground and reference screw electrodes were placed above the cerebellum. Neck muscle electrodes were implanted bilaterally, and bipolar referencing was used for EMG. A custom-made EEG/EMG connector was fixed on the skull with dental cement for sleep recordings.

Surgical procedure for Ca²⁺ imaging. The surgical procedures were previously described (Ziv et al., 2013). First, 500 nL of the viral vector AAV5-CaMKII-GCaMP6f (prepared by University of North Carolina Vector Core) was injected into the CA1 pyramidal layer at the following coordinates: −2.1 mm AP, −1.5 mm ML, and −1.3 mm dorsoventral (DV) to bregma. The skin was sutured and disinfected using Betadine solution. Two weeks after virus injection, a glass guide tube was implanted directly above CA1. For this, a trephine drill was used to remove a circular part of the skull located posterolateral to the viral injection site, and the dura, cortex and the hippocampal commissures above the CA1 were removed by suction with a 29 gauge blunt needle while constantly washing the exposed tissue with sterile PBSx1. A glass guide tube was then implanted above the CA1 stratum pyramidal. A recording electrode (bipolar stainless steel; 0.127 mm diameter) was slowly lowered through a hole that was drilled adjacent to the guide tube into CA1 (1 mm DV). The space between the skull and the optical guide tube was sealed using a low toxicity silicone adhesive (Kwik-Sil, WPI surgical instruments) and the remaining exposed area of the skull was covered with dental cement and dental acrylic. A metal bar was added to the posterior aspect of the construct in order to head fix the animal when needed.

Ca²⁺ imaging in behaving mice. For time-lapse imaging in behaving mice we used an integrated miniature fluorescence microscope (nVista 3.0, Inscopix) as previously described (Zarhin et al., 2022). At least two weeks after the glass guide tube implantation, we inserted a microendoscope consisting of a metal guide cannula (˜3.1 mm length, 1.8 mm outer diameter) and a single gradient refractive index lens (4.0 mm length, 1.0 mm diameter) into the implanted glass tube and examined Ca²⁺ indicator expression in the operated mice (Inscopix data acquisition software, Inscopix). We then affixed the microendoscope within the glass guide tube using ultraviolet-curing adhesive (Flow-It A3, Pentron). Next, we attached the miniature microscope's magnetic base plate to the dental acrylic surface with the ultraviolet-curing adhesive. At least a day later, the mice were habituated wearing the miniature microscope in their home cage for 30 minutes for 4-5 days. To record mouse behavior in the home cage, we used an overhead monochrome camera (GigE Vision, Basler AG), which we synchronized with the miniature microscope. Ca²⁺ imaging was performed at 10 Hz. Imaging sessions consisted of 18-min-long trials.

Ca²⁺ imaging in behaving and anesthetized mice. For time-lapse imaging in behaving mice we used an integrated miniature fluorescence microscope (nVistaHD 2.0 or n Vista 3.0, Inscopix) as previously described (Ziv et al., 2013). At least two weeks after the glass guide tube implantation, we inserted a microendoscope consisting of a metal guide cannula (˜3.1 mm length, 1.8 mm outer diameter) and a single gradient refractive index lens (4.0 mm length, 1.0 mm diameter) into the implanted glass tube and examined Ca²⁺ indicator expression in the operated mice (Inscopix data acquisition software, Inscopix). We selected for further imaging only those mice that exhibited homogenous GCaMP6f expression throughout the field of view, without signs of injury or inflammation (˜85% mice passed the selection criteria). For the selected mice, we then affixed the microendoscope within the glass guide tube using ultraviolet-curing adhesive (Flow-It A3, Pentron). Next, we attached the miniature microscope's magnetic base plate to the dental acrylic surface with the ultraviolet-curing adhesive. A day later, the mice were habituated for 4-5 days to freely explore a 50×50 cm open-field, with the miniature microscope. Before the beginning of each session, the open-field was thoroughly cleaned with a 70% ethanol solution. To record mouse behavior, we used an overhead monochrome camera (GigE Vision, Basler AG), which we synchronized with the miniature microscope. Behavioral analysis was performed with ToxTrac software (Rodriguez et al., 2018). Ca²⁺ imaging was performed at 10 Hz. Imaging sessions consisted of 5-15-min-long trials, while the inter-trial interval was ≥15 min.

For Ca²⁺ imaging sessions during different types of anesthesia, the mice were head-fixed to a stereotactic device before the application of anesthesia and their body temperature was maintained by a temperature controller (FHC, 40-90-8D). LFP activity in the CA1 was recorded throughout the duration of anesthesia and Ca²⁺ measurements were taken once a stable LFP pattern was observed, 60 minutes following anesthesia induction. For the inhalatory anesthetic isoflurane, experiments were performed under 1.0% and 1.5% isoflurane, combined with oxygen (100%). Ketamine (100 mg/kg), supplemented with xylazine (8 mg/kg), were injected i.p. Medetomidine (0.3 mg/kg) was injected i.p and stopped using the synthetic α₂ adrenergic receptor antagonist atipamezole (1 mg/kg, i.p). The drugs were diluted with sterile PBS before injection.

For Ca²⁺ imaging during sleep-wake cycle, two stainless-steel wires were inserted to either side of neck muscles, and referenced bipolarly, to measure EMG activity. Imaging analysis was performed during extended (≥5 min with less than 30 sec interruption by the other state) bouts of active wakefulness or NREM sleep based on LFP/EMG recordings during 6 hours of the light cycle (10 AM-4 PM). 5 kHz noise in the LFP/EMG recordings generated by electronic focus of the n Vista3 microscope was filtered out by a band-pass filter (1-300 Hz). Mice were habituated to the recording chamber for several days before the experiment.

Ca²⁺ imaging and LFP recording in anesthetized mice. For Ca²⁺ imaging and LFP recording sessions during anesthesia, mice were placed in an induction chamber connected to isoflurane vaporizer (Isotec 5), gas flow rate was turned on to 0.8 LPM (litre per minute) and the vaporizer was set at 5% isoflurane. When mouse' breathing slowed down and became rhythmic, it was moved to a physiological monitoring system (75-1500 Harvard Apparatus) and isoflurane level was reduced gradually and slowly to 1.5%. Respiration and heart rate were monitored and analyzed by the physiological monitoring system. To achieve a steady and consistent anesthetic depth, isoflurane level was slightly adjusted between 1.3%-1.7% when needed in order to maintain a stable LFP activity pattern of 0.2-0.5 BSR and respiratory rate of 45-90 RPM throughout the duration of the anesthesia. Temperature was also maintained by the device at 37° C. throughout the procedure.

LFP activity in the CA1 was recorded throughout the duration of anesthesia and Ca²⁺ measurements were taken once a stable LFP pattern was observed, ˜120 minutes following anesthesia induction. Experiments were performed under 1.3-1.7% isoflurane, combined with oxygen (100%).

Ca²⁺ imaging data analysis. We pre-processed the raw imaging data using a commercial software (Inscopix data processing software, Inscopix). Raw data was spatially down sampled by a factor of 2, cropped to 1200 by 840 μm rectangle, motion corrected and exported as TIFF file. Single neurons and their calcium signals for each data set were extracted using the constrained non-negative matrix factorization algorithm for endoscopic recordings (CNMF-E, Zhou et al., 2018). CNMF-E algorithm can reliably extract cellular signals from one-photon calcium imaging data sets by parallel denoising, deconvolution and demixing. The maximal diameter of neurons in the imaging plane was set to 13 pixels (gSiz), and the width of the gaussian kernel, which can approximate the average neuron shape was set to 3 pixels (gSig). Besides pre-processing spatial down sampling, no further spatial or temporal down sampling was performed (ssub=1, tsub=1 respectively). Neurons with spatial overlap ratio greater than 0.85, temporal correlation of calcium traces greater than 0.85 and centroid distance less than 1 pixel (dmin) were merged. Region of interest (ROIs) that had a minimum peak-to-noise ratio for a seeding pixel of 8 (min_pnr) and minimum spike size of 5 (smin, when the value is negative, the actual threshold is=smin*noise level) were extracted. The foopsi method was used for deconvolution (Pnevmatikakis et al., 2016). These parameters were used for all data sets analyzed in the paper. Due to one-photon background fluorescence fluctuations, motions artifacts and segmented dendrites, some of the ROIs detected by CNMF-E cannot be considered as true cells, but false positive detections. Therefore, we have inspected the registered ROIs spatial footprints and excluded them based on the following exclusion criteria: minimum peak-to-noise ratio of 8, 300>size>30 pixels, and minimum circularity estimate of 0.5 (1 equals a perfect circle). Filtered data sets were further manually inspected and verified by the experimenter (˜5-10% cells were excluded per data set). CNMF-E “S” output was used as the inferred spiking activity (firing rate) obtained from the denoised (“C”) scaled version of DF (“C_raw”). “Firing rate” of a cell was defined as it summed inferred spiking activity (obtained from the “S” output) throughout the recording session, divided by the recording time. Single-cell and population analysis were performed with custom MATLAB functions.

Anesthetic depth analysis. Anesthetic depth was modulated by isoflurane level: 1% for moderate anesthesia and 1.5% for deep anesthesia. For vital physiological measurements, mice were placed in an induction chamber connected to isoflurane vaporizer (Isotec 5), gas flow rate was turned on to 0.8 LPM and the vaporizer was set at 5% isoflurane. When mouse' breathing slowed down and became rhythmic, it was moved to physiological monitoring system (75-1500 Harvard Apparatus) and isoflurane level was reduced gradually and slowly to either 1.0% or 1.5%. Respiratory rate was monitored and analyzed by the physiological monitoring system. Temperature was also maintained by the device at 37° C. throughout the procedure. No changes in respiratory rate were found between all the genotypes used in the paper at 1.0% and 1.5% isoflurane.

For detecting burst-suppression (deep anesthesia), raw LFP recordings were divided to 500 ms bins and for each bin three parameters were calculated: (1) maximum absolute value, (2) standard deviation, and (3) the first principle component of the spectrogram calculated between 1-100 Hz. Each of these parameters showed a bimodal lognormal distribution and thus were used to classify the bins as periods of bursts or suppression using a gaussian mixture model. Epochs of bursts were merged if the inter-burst interval was less than 2 seconds and were accepted as bursts only if their duration was greater than 2 seconds. These criteria were empirically selected as they increased the algorithm's robustness. Further, manual scoring revealed that indeed >95% of bursts were longer than 2 seconds. BSR was calculated in ˜1 min bins (52.428 s) as the fraction of time in suppression. Overall, this algorithm demonstrated >92% accuracy when compared to manual scoring of two data sets from different genotypes. Relative delta power was calculated in ˜1 min bins as the power spectral density (PSD) between 1-4 Hz divided by the broadband PSD in 1-100 Hz. This ratio was z-scored and normalized between 0 and 1. Epochs of deep anesthesia were defined as 0.3<BSR<0.8 and epochs of moderate anesthesia were defined as BSR<0.3 and relative delta power>0.5. These epochs were merged if the inter-epoch interval was less than 1 min and were accepted only if their duration was greater than 1 minute.

c-Fos staining. 30-60 min post-GA, mice were transcardially perfused with cold PBS followed by 4% paraformaldehyde (PFA) in PBS. The brains were extracted, postfixed overnight in 4% PFA at 4° C., then rinsed 3 times with 0.1×PBS and kept in PBS. Brains were sectioned to a thickness of 50 μm using Leica microtome, then washed 3 times for 5 minutes in 1xPBS, then in PBST (0.1% TritonX-100). Free-floating sections were washed in PBS, incubated for 1 h in blocking solution (1% BSA and 0.3% Triton X-100 in PBS) and incubated with the primary c-Fos antibody (rabbit, Synaptic Systems, Cat.No. 226 003; 1:10,000) for five nights at 4° C. Sections were then washed with PBS and incubated for 2 h at room temperature with secondary antibody (goat anti-rabbit, DyLight 488, Jackson Laboratories; 1:600) in 1% BSA in PBS. Finally, sections were washed in PBS, mounted on slides with DAPI mounting medium (Sigma, F6057) and sealed.

Detection of pathological spikes in fAD models. High-voltage pathological spikes were detected by setting a threshold of 10 z-scores above and below the mean voltage during the suppression epochs of the recording and accepted only if their peak-to-peak value within 30 ms was greater than 10 z-scores. A dead-time of 50 ms was set to assure the same spike was not counted twice. Subsequently, all spikes were manually inspected and approved.

Spatial working memory test. Spatial working memory was examined using a continuous variation of the T-maze called Delta-maze (Wood et al., 2000). In sum, mice were required to traverse the central arm of a delta-shaped maze and alternate between left and right turns during subsequent trials. Correct trials were reinforced with a drop of condensed milk at the edge of the selected side arm. After entering one of the side arms, retracing was prevented by hinged doors such that mice could initialize a new trial only by returning to the base of the central arm via a connecting arm. At the base, mice were confined for a specified delay before the door to the central arm was opened and a new trial commenced. The first trial of each session contained a reward in both side arms of the maze. Prior to the onset of training, mice were habituated to the apparatus for 15 m a day for 5 consecutive days. During training, mice performed 10 trials of the task with a 10 s delay. During testing, the delay varied between days from 10 to 180 s. To facilitate learning and assure consistent motivation when performing the task, food intake of mice was restricted such that their weight was kept at 85-90% relative to ad libitum feeding. Success rate was defined as the percentage of correct alternations relative to the number of trials.

Single-unit surgery, data acquisition and analysis. Mice were implanted with a costume-made microdrive (custom printed circuit board and drive by Rogat, Carmiel, Israel) along with neck muscle electrodes implanted for electromyography (EMG). The microdrive contained a moveable assembly of 4 tetrodes (17-um, platinum 10% iridium, California Fine Wire) and was connected to the recording setup via an Omnetics headstage connector (Connector Corporation, Minneapolis MN, USA). Two holes were drilled in the skull: one in the frontal bone plate for a screw serving as ground; the second hole for the electrodes implanted in the parietal cortex (1.94 mm posterior of bregma; 1-1.2 mm medial lateral axis; 1-1.2 mm dorsal ventral axis). After 7 days of monitored recovery, subsequent downward movements of the microdrive were made in 25- to 50-μm increments over 24-hr intervals until approaching the CA1 pyramidal cell layer, recognized by the appearance of multiple high-amplitude units and spontaneous ripple events. At the end of the experiment, a small electrolytic lesion was made (30 μA for 20 sec) under anesthesia. Two days after, histology procedure was performed to verify electrodes location as described (Weiss et al. (2017), Consistency of Spatial Representations in Rat Entorhinal Cortex Predicts Performance in a Reorientation Task, Current Biology 27, 3658-3665.c3654).

Animals were recorded during the first half of the light cycle in a familiar environment (home cage). Raw data were sampled at 24 KHz using a Neurophysiology Workstation (RZ5D base processor and PZ5 NeuroDigitizer amplifier, Tucker-Davis Technologies Inc). Offline, spikes were detected as crossings above four STDs of the whitened data (Pachitariu et al., 2016, Fast and accurate spike sorting of high-channel count probes with KiloSort, Advances in neural information processing systems 29, 4448-4456). Spike waveforms (40 samples symmetrical around the peak) were semi-automatically clustered using KlustaKwik (Kadir et al., 2014) followed by manual inspection using Klusters (Hazan et al., 2006, Klusters, NeuroScope, NDManager: A free software suite for neurophysiological data processing and visualization. Journal of Neuroscience Methods 155, 207-216). All subsequent data analysis was done using MATLAB (Mathworks, Natick MA). Clusters were defined as well-isolated single units and included in the analysis only if they fulfilled the following criteria: (1) the presence of a refractory period (less than 0.5% of inter-spike intervals<2 ms (Middleton et al., 2018, Altered hippocampal replay is associated with memory impairment in mice heterozygous for the Scn2a gene. Nature Neuroscience 21, 996-1003); (2) an isolation distance>10 (Tanaka et al., 2018, The hippocampal engram maps experience but not place, Science 361, 392-397), (3) MFR>0.05 Hz during active wakefulness; and (4) a stable firing rate during the recording period (Styr et al., 2019). Using the CellExplorer open source software (Petersen et al., 2020, CellExplorer: a graphical user interface and a standardized pipeline for visualizing and characterizing single neurons, bioRxiv, 2020.2005.2007.083436), clusters were separated to regularly-spiking and fast-spiking units semi-automatically by inspecting their waveform, autocorrelations and burst index (Royer et al., 2012, Control of timing, rate and bursts of hippocampal place cells by dendritic and somatic inhibition. Nature Neuroscience 15, 769-775).

To compare of MFRs between active wakefulness and NREM sleep, we normalized the differences in response magnitudes by the maximum absolute value, to produce an unbiased gain factor (Sela et al., 2020, Sleep Differentially Affects Early and Late Neuronal Responses to Sounds in Auditory and Perirhinal Cortices. The Journal of Neuroscience 40, 2895-2905) as follows:

$$\% Gain \text{?} = \frac{a - b}{max ( \vert a \vert , \vert b \vert )} \times 100$$

$$\text{?} \text{indicates text missing or illegible when filed}$$

where a is the MFR in NREM sleep and b is the MFR in active wakefulness. Therefore, a negative gain factor indicates a reduced response during NREM sleep, and vice versa. Gain factors across neurons are summarized as median, and [lower and upper] 95% CIs around the median.

Scoring of vigilance stages. Vigilance states were manually scored in 5-s epochs based on visual inspection of frontal EEG or CA1 LFP and EMG as previously described (Barger et al., 2019, Robust, automated sleep scoring by a compact neural network with distributional shift correction. PLOS ONE 14, e0224642). Wakefulness was defined by low-amplitude, high-frequency EEG/LFP activity, recorded in the frontal lobe, and high EMG activity. Wakefulness was further divided to states of active wake (exploration, grooming, eating) and quiet wake based on video and EMG recording. NREM sleep was defined by high-amplitude, low-frequency EEG/LFP, and reduced EMG tone. REM sleep was defined by low-amplitude, high-frequency EEG/LFP, dominated by theta activity, and with flat EMG. States transitions and artifacts (<3% of recording time) were excluded from further analysis. Spectrograms were constructed by Fourier frequency transformation (1 second bins) of the EEG signal.

Multi-electrode array (MEA). Postnatal hippocampal cultures were plated on MEA plates containing 120 titanium nitride (TiN) electrodes, in addition to 4 internal reference and 4 ground electrodes (Styr et al., 2019). Each electrode has a diameter of 30 μm and electrodes are arranged in a 12×12 grid (sparing 6 electrodes in each corner), spaced 100-200 μm apart on average (Multi Channel Systems, 120MEA200/30iR-Ti). Data acquisition was done in 3-weeks-old cultures using a standard MEA2100-Systems with a hardware filter cut-off of 3.3 kHz and sampling rate of 10 kHz per electrode. Recordings were carried out under constant 37° C. and 5% CO₂ levels.

Data analysis: Raw data was filtered, offline, at 200 Hz using a Butterworth high-pass filter. Spikes were then detected, offline, using MC_Rack software (Multi Channel Systems) based on a fixed threshold set to between 5-6 standard deviations from mean. Twenty minutes of each hour (that were previously shown to reliably represent the MFR of the entire hour, were used for analysis to reduce processing time and analyzed using custom-written scripts in MATLAB (Mathworks) as previously described (Slomowitz et al., 2015). Channels with unstable (>30% change of MFR) baseline recordings during 3-4 hours prior to a perturbation were excluded from the analysis.

Electrophysiology in slices. Acute hippocampal slices (coronal, 400 μm) were prepared from WT and APP/PS1 mice. Slices were transferred to a submerged recovery chamber at 32° C. containing oxygenated (95% O₂ and 5% CO₂) artificial cerebrospinal fluid (ACSF) for 1 h before the experiment. The ACSF contained, in mM: NaCl, 125; KCl, 2.5; CaCl₂, 1.2; MgCl₂, 1.2; NaHCO₃, 25; NaH₂PO₄, 1.25; glucose, 25. fEPSPs were recorded in acute hippocampal slices with a glass pipette containing Tyrode solution (1-2 MΩ) from synapses in the CA1 using a MultiClamp700B amplifier (Molecular Devices). Stimulation of the Shaffer Collateral (SC) pathway was delivered through a glass suction electrode (10-20 μm tip) filled with Tyrode. Data were analyzed using pClamp10 (Molecular Devices).

Metabolic profiling. For determining orotate concentration in the hippocampus of WT and APP/PS1 mice, mice were anesthetized by 1.5% isoflurane and hippocampi were dissected and then homogenized by Bullet Blender homogenizer (Next Advance) at 4° C. with methanol: acetonitrile: water (5:3:2 ratio) solution at 40 mg/ml concentration. Homogenized samples were centrifuged for 15 min at 4° C. at 16,000×g. The supernatants were transferred to glass HPLC vials and stored at −80° C. Metabolic profiling was done using an Ultimate3000 UHPLC system (Dionex, Thermo Scientific) coupled to a Q-Exactive Plus mass spectrometer (Thermo Scientific). Metabolite separation was done using a 49 min gradient of buffer A (95% acetonitrile) and buffer B (50 mM ammonium carbonate, pH 10, 5% acetonitrile) using SeQuant ZIC-pHILIC column (Merck; 150 3 2.1 mm, 5 mm) coupled to a SeQuant ZIC-pHILIC guard column (Merck; 20 3 2.1 mm, 5 mm) with flow rate of 0.1 ml/min. Data were acquired by switching between negative and positive polarity modes using full MS scans. Identification of orotate was done using LCquan software (Thermo Scientific) based on external standards.

Protein extracts and ELISA for Aβ. After transcardial perfusion with cold PBS hippocampus were dissected, snap-frozen in liquid nitrogen, and stored at −80° C. until use. Proteins from both hippocampus of 5-month-old APP/PS1 and APP-KI mice were sequentially extracted in a 2-step procedure. Tissue (˜0.2 mg/mL wet weight) was homogenized using a mechanical homogenizer in 50 mMTris-HCl, pH 8.0, 150 mM NaCl, complete proteinase inhibitor cocktail (Roche) and 10 mg/mL Pepstatin A, centrifuged at 100,000×g for 1 hour at 4° C., and supernatants containing the soluble fraction were collected, stored at −80° C., and used for quantification of soluble Aβ. Pellets were resuspended, incubated on ice for 1 h and sonicated in 6 M guanidine-HCl, 50 mM Tris-HCl, pH 7.4, centrifuged at 100,000×g for 1 h at 4° C., and supernatants containing membranous fraction and insoluble aggregates were collected, stored at −80° C., and used for quantification of insoluble Aβ. Protein quantification was performed using Bradford method. The levels of soluble Aβ40 and Aβ42 in mice hippocampus extracts were detected using sandwich ELISA kits (Wako, Japan), according to the manufacturer's instructions. Concentrations of soluble and insoluble Aβ are expressed in pM per μg of total soluble or insoluble protein, respectively.

fEPSP measurements in vivo in anesthetized mice. Operated mice recovered in their home cage for at least a week following electrodes implantation. At the beginning of the experiment, mice were habituated for a few days to the experimental device that was composed from a running wheel and metal bar in which the mice were head fixed into throughout the experiment with a screw. For fEPSP measurements in CA3-CA1 synaptic connections during anesthesia, head fixed mice were anesthetized (5% isoflurane for induction, 1.5% isoflurane for the maintenance) and their body temperature was maintained by a temperature controller (FHC, 40-90-8D). Mice LFP activity was recorded throughout the duration of the anesthesia, and fEPSP measurements were taken once a stable LFP pattern was observed, 60 minutes from the induction of the anesthesia.

Analysis of network pattern. For network pattern analysis, we created a sequences of inferred spike times per cells that were identified using the CNMF-E algorithm. We projected this data onto a single timeline to produce an ordered time sequence that demonstrates individual cell firing activity. Next, we measured the relative synchronized activity per time bin of 100 ms, defined as:

(Relative Synchronization)n=Σ(Active cells)n(Total cells detected),

in which “n” equals to the time bine analyzed. Synchronization vector peaks that were higher than the 90th percentile in exploration sessions, or higher than 0.05 in anesthesia sessions were defined as network bursts. Threshold of 0.05 was chosen for anesthesia sessions since it gave the most reliable network bursts detection based on visual inspection and verification of the data by the experimenter. Similar results were obtained when testing different thresholds (e.g., 0.1 and 0.15, data not shown). We than analyzed the inter-burst interval, number of cells that were active in each network burst, and number of spikes that comprised each network burst. 20 random samples were taken from each mouse for pooled analysis.

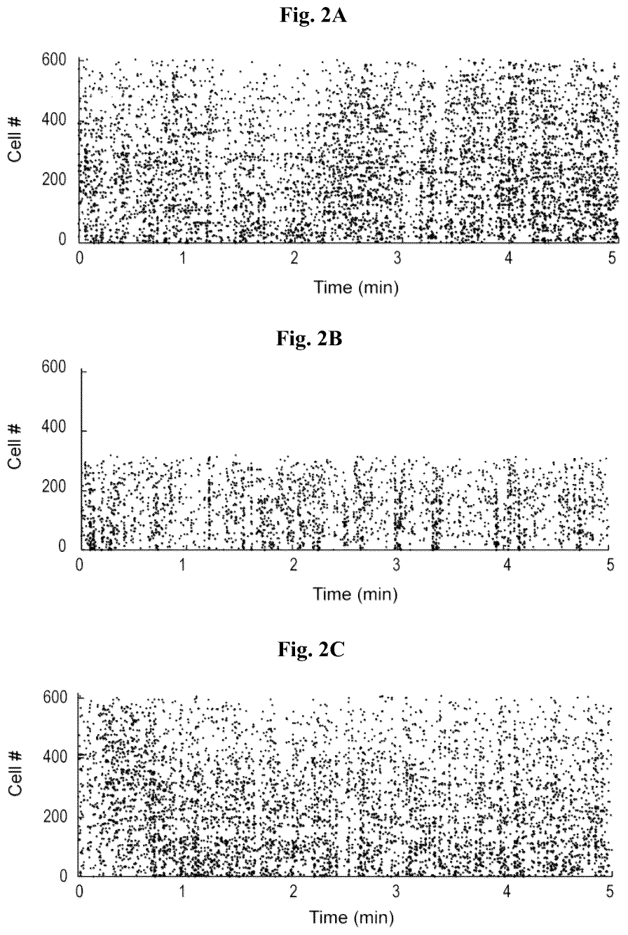

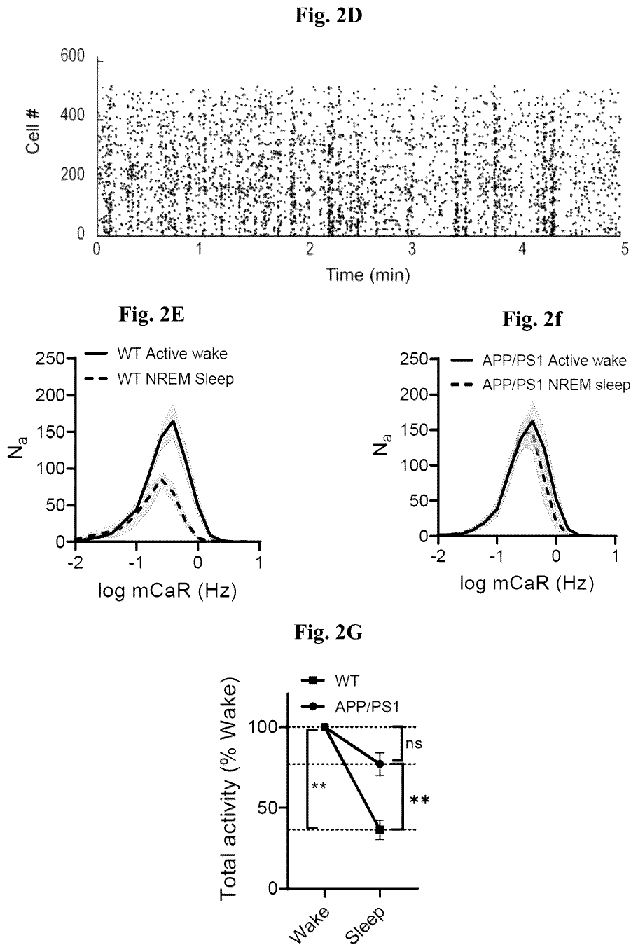

Microstate clustering using t-SNE/WS algorithm. We identified microstates of CA1 hippocampal neuronal population using the un-supervised nonlinear embedding method, t-Distributed Stochastic Neighbor Embedding (Laurens van der Maaten, 2008) (t-SNE) and an image processing algorithm-watershed (WS) combined into t-SNE/WS clustering algorithm. The input for the clustering algorithm was the inferred, de-convoluted spiking patterns based on Ca²⁺ signals provided by the CMNF-E algorithm. The analysis was performed on active frames that included at least 2 co-active neurons as described in an earlier study (Wenzel et al., 2019), with some modifications related to initial dimensionality reduction and perplexity value calculation. For noise reduction, we used an initial dimensionality reduction based on principle component analysis (PCA). The principal components>30^{th} percentile of the derivative of the cumulative explained variance were selected and the data was projected upon them to create a lower dimensional space (post-PCA space). The perplexity parameter for the t-SNE was calculated as # of active frames in each experiment. Using the calculated perplexity value and initial dimensionality reduction, t-SNE was applied with 1000 iterations to produce a robust 2D embedding space that could be analyzed and visualized. To create density maps, the embedded points on the 2D map, representing patterns of coactive cells per frame, were smoothed by a Gaussian kernel with a standard deviation equal to 1/60 of the maximum coordinate in the embedded space. To turn patterns of coactive neurons into separated microstates, we used a watershed algorithm (Wenzel et al., 2019) on the density map. A microstate was defined as a region created by the WS algorithm which contains a cluster of similar co-activity patterns based on Ca²⁺ imaging data analyzed in FIG. 2. The size of each microstate depends on the frequency of a specific pattern during the recording session. For clustering validation, we used an inter-and intra-cluster correlation score, calculated based on PCA analysis. To calculate the number of microstates in a randomized data, we permuted the activity in each frame, so the number of co-active cells in each frame was maintained, while the cells' identity was different.

Clustering microstates using affinity propagation clustering. We used the affinity propagation cluster (APC) algorithm (Frey and Dueck, 2007, Clustering by passing messages between data points. Science 315, 972-976) as an additional microstate clustering method. It is an efficient clustering algorithm that takes as inputs the similarities between pairs of observations in the dataset (frames, in our case), and finds exemplars and the observations they represent by exchanging real-valued messages between data points. We used the MATLAB (Mathworks) ‘apcluster’ function made available by the authors. We used the post-PCA correlation between frames as the measure of similarity used by the algorithm. We also used convits=10 and a preference equal to the median similarity of the dataset.

Microstate analysis using hierarchical clustering. We used MATLAB's ‘linkage’ function to build a hierarchical bottom-up tree with weighted correlation metric based on the PCA data. ‘Cluster’ function was used to cluster the tree with distance cutoff of 0.75.

Contextual fear conditioning test. Contextual fear conditioning (CFC) was performed in a 25.5×25.5×36 cm chamber with electric grid floor. For the conditioning session, the mice were placed in the CFC apparatus for 2.5 min, and then a pure tone (2.9 kHz) was introduced for 20 sec, followed by a 0.6 mA foot shock for 2 sec. Another tone and shock were introduced again after 1 min, and then, 30 sec after the second shock, the mice were returned to their home cages. The context in which the mice received foot shock consisted of green lighting, white noise, vanilla scent and square perspex walls. To test contextual fear memory, mice were placed back in the familiar context for 5 min. The mice were later placed in a novel context for 2.5 min, consisted of white lighting, rum scent and round walls. The apparatus was cleaned between every session using 70% Ethanol and Virusolve. Automatic freezing detection of the recorded videos was conducted using the EthoVision software.

Histological verifications. To check the expression of AAV5-CaMKII-GCaMP6f in the CA1 and the precision of the injection location and micro endoscope implantation site, we used 2-photon microscope (LSM 7 MP, Zeiss) to image the pyramidal layer in hippocampal slices. Chameleon Ti:Sapphire laser system with a 80 MHz repetition rate was used to excite the sample. The excitation wavelength was 920 nm. Emission light was filtered by 500-550 nm band-pass filter. Three-four weeks after injection of the virus or at the end of the behavioral experiments, we perfused mice with phosphate-buffered saline (PBS) followed by cold 4% paraformaldehyde (PFA). We then removed the perfused brains and kept them in PFA solution for 24 h. 70-μm coronal slices were obtained from the perfused brains using a Leica VT1200 vibrating microtome and stored in PBS for further imaging. Validation of the injection site, implanted micro endoscope location and viral expression was obtained by imaging in 2-photon microscope.

Data collection and analysis. While data collection was not performed blind to the condition of experiment, investigator was blind to these conditions throughout much of the analysis, as spikes/calcium signals were automatically detected, and their manual inspection happened without any knowledge of experimental conditions.

Statistical analysis. Error bars shown in the figures represent SEM. All the experiments were repeated at least in three different animals and repeated within the animal at least twice. Statistical significance was assessed by unpaired or paired Student's t-tests, Mann-Whitney U-tests, one-way analysis of variance (ANOVA), or two-way ANOVA, where appropriate (multiple comparison tests are specified in figure legends). Normality was assessed using the Shapiro-Wilk test. For non-normal distributions, differences between groups were tested with Wilcoxon signed-rank test for paired data and Mann-Whitney test for unpaired data. Correlation was assessed using Spearman test. Comparison of distributions was performed by Kolmogorov-Smirnov test. Statistical analysis was performed using Prism 9.0 GraphPad. The statistical test, p value and the number of cells/mice that went into the calculation are reported in figure legends. Significance was declared at p<0.05 and all tests were two-sided.

### EXAMPLE 1

### CA1 Population Activity is Normal During Active Wakefulness in APP/PS1 Mice

First, we characterized amyloid homeostasis and hippocampus-dependent memory in early-stage (4-5-month-old) APP/PS1 mice. APP/PS1 (APP_{Swe}/PS1ΔE9) mice are double transgenic mice expressing a chimeric mouse/human APP (Mo/HuAPP695swe) and a mutant human presenilin 1 (PS1-dE9), both directed to CNS neurons. Both mutations are associated with early-onset AD. These mice display pathologically increased Aβ40, Aβ42 and Aβ42/Aβ40 ratio for both soluble and insoluble Aβ fractions (data not shown). Hippocampus-dependent memory functions, such as spatial working memory and context-dependent fear memory were unimpaired at this age (data not shown). We consider this stage as pre-symptomatic, although we acknowledge that fAD models do not recapitulate all the changes observed during the pre-symptomatic stage in AD patients (such as accumulation of tau pathology and hippocampal volume loss). Notably, memory decline was evident at a more advanced disease stage, in 9-month-old APP/PS1 mice (data not shown).

To analyze neuronal activity in large neuronal populations in freely behaving mice, we employed wide-field, head-mounted miniaturized microscopes (Ziv et al., 2013). This technique enables tracking of Ca²⁺ dynamics with single-neuron resolution as a proxy for neuronal activity (Chen et al., 2013). The integrated miniaturized microscope allows for high-speed and large-scale longitudinal recordings of Ca²⁺ dynamics from genetically defined neuronal populations in various deep brain structures, including the hippocampus, in freely behaving mice. Utilizing this method, we monitored activity patterns of thousands of CA1 pyramidal neurons and analyzed active neurons at each session during active wakefulness. We imaged fluorescence generated by a genetically encoded Ca²⁺ sensor GCaMP6f (Chen et al., 2013) expressed in excitatory CA1 pyramidal neurons under the CaMKIIα promoter. Experiments were performed in 6 APP/PS1 mice (3,973 neurons total) and 6 wild-type (WT) littermates (3,846 neurons total), while they explored a familiar open field. Imaging was performed daily at regular hours during light phase to avoid circadian effects. Firing rate of single cells was approximated from the Ca²⁺ event rates using the CMNF-E method (Zhou et al., 2018) optimized for one-photon Ca²⁺ imaging. No detectable changes in Ca²⁺ event rate distributions of CA1 neuronal populations were found between WT and APP/PS1 mice in active wakefulness (FIG. 1A). Mean Ca²⁺ event rate (mCaR) per cell was very heterogeneous in both WT and APP/PS1 mice (FIG. 1A). No differences were observed in physical activity of WT vs. APP/PS1 mice during exploration of a familiar environment (data not shown). Detailed analysis of all recorded neurons revealed no difference in the median mCaR (FIG. 1B) or in the number of active neurons (Nₐ, FIG. 1C), resulting in similar total level (Nₐ*mCaR) of CA1 activity between WT and APP/PS1 mice (FIG. 1D). Also, no differences were detected in the statistical attributes of CA1 network activity between behaving WT and APP/PS1 mice (data not shown).

Although somatic Ca²⁺ signals in neurons are used as a proxy of spiking activity, whether spike-to-Ca²⁺ transfer function is preserved under variable experimental conditions is unknown and the sensitivity of microendoscopy for Ca²⁺ transients evoked by single action potential remains to be improved. Therefore, we used chronically implanted tetrodes to directly record single-unit spiking activity in behaving WT and APP/PS1 mice. The recordings of CA1 firing rates were performed during the same hours of light phase, as Ca²⁺ recordings, in a familiar environment (home cage). Vigilance state analysis was performed by analyzing LFP and EMG in the same mice with single-unit recordings, and in a separate batch of mice by analyzing EEG/EMG and video recordings. Criteria for clustering of single units and separation of regular spiking (RS), putative pyramidal neurons from fast-spiking (FS), putative interneurons were based on a previous analysis (Watson et al., 2016) and optimized for our recording conditions. Our results show no difference (p=0.82) in MFR of the CA1 population of RS neurons between WT (2.01±0.195 Hz, 103 units, 4 mice) and APP/PS1 mice (1.88±0.15 Hz, 98 units, 4 mice) in active wakefulness (FIG. 1E). Furthermore, no difference was observed in MFRs of CA1 FS neurons (data not shown). The duration of active wake state and physical activity of mice (data not shown) were similar between groups. Thus, two independent recording methods with single-neuron resolution—Ca²⁺ microendoscopy and single-unit electrophysiology—demonstrate that CA1 MFRs are unaltered during active wakefulness in early-stage APP/PS1 mice.

Finally, we recorded the extracellular field excitatory postsynaptic potentials (fEPSPs) in the CA3-CA1 pathway in awake WT and APP/PS1 mice. No changes were observed in CA3-CA1 synaptic transmission and short-term synaptic plasticity between awake WT and APP/PS1 mice (FIG. 1F-G). Overall, these results demonstrate that CA1 firing rates, CA3-CA1 synaptic transmission and short-term plasticity are not impaired during active wakefulness in APP/PS1 mice, before the onset of memory decline.

### EXAMPLE 2

### Dysregulation of CA1 MFRs During NREM Sleep in APP/PS1 Mice

Next, we asked whether local homeostatic mechanisms underlying down-regulation of CA1 neuronal activity by NREM sleep are maintained in the early-stage fAD mice. To analyze regulation of CA1 neuronal activity by sleep, we imaged CA1 dynamics, in parallel with LFP/EMG recordings, during the sleep-wake cycle of mice. The recordings were performed in 5 WT and 4 APP/PS1 mice during the same hours of the light cycle. Ca²⁺ imaging was analyzed during periods of wake-dense episodes dominated by active wakefulness (data not shown) and sleep-dense episodes dominated by NREM sleep periods (data not shown). As expected, WT mice showed ˜60% reduction in total CA1 activity during NREM sleep due to a reduction in the number of active neurons and the mean Ca²⁺ event rate, in comparison to active wakefulness (FIG. 2A-B, E). In contrast to WT mice, neither the number of active neurons, nor the mean Ca²⁺ event rate was significantly changed during NREM sleep of APP/PS1 mice, resulting in similar total activity levels during active wake and NREM sleep periods (FIG. 2C-D, F). Thus, the typical negative regulation of CA1 population activity by NREM sleep was significantly diminished in APP/PS1 in comparison to WT mice (FIG. 2G). These results indicate that homeostatic regulation of CA1 MFRs is impaired in a state-dependent manner in APP/PS1 mice.

### EXAMPLE 3

### Local Dysregulation of CA1 Firing Rates During NREM Sleep Precedes Global Deterioration of SWA in APP/PS1 Mice

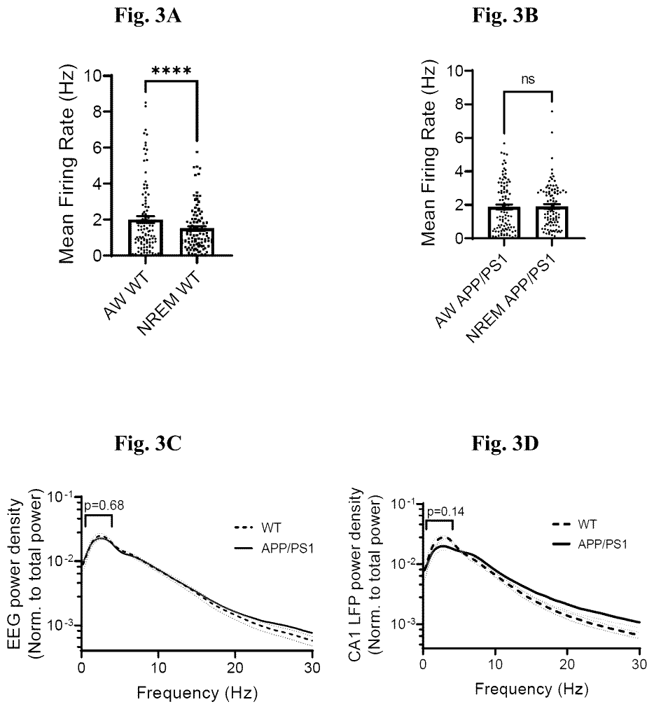

Next, we asked whether local homeostatic regulation of firing rates by NREM sleep is impaired because of the deterioration of slow wave oscillations, as reported in AD patients (Lucey et al., 2019; Mander et al., 2015) and in fAD mouse models after the onset of cognitive decline (Kent et al., 2018). To compare CA1 firing rates between WT and APP/PS1 mice, we used single-unit recordings. In WT mice, MFRs of CA1 RS neurons were decreased on average from 2.01±0.195 Hz to 1.51±0.12 Hz during NREM sleep in comparison to active wakefulness (FIG. 3A, 4 mice 103 single units). We proceeded to quantitatively compare MFR of each unit in active wakefulness and NREM sleep across the entire dataset. A sub-population of CA1 high-rate excitatory neurons (defined as neurons whose MFRs during active wakefulness were above the median of 1.4 Hz) showed attenuation with median gain factor of −21% in NREM sleep (45% of all the units, p<0.0001, data not shown). However, a sub-population of low-firing neurons (defined as neurons whose MFRs were below the median) displayed heterogeneous gain factors with a tendency towards a small increase (+1.7%, p=0.055, data not shown), confirming the results of a previous study conducted in the cortex (Watson et al., 2016). This physiological regulation of firing rates in CA1 pyramidal neurons by NREM sleep was disrupted in APP/PS1 mice. On average, MFRs of the CA1 network were not different (1.88±0.15 Hz in AW vs. 1.91±0.13 Hz in NREM, p=0.53) between active wakefulness and NREM sleep (FIG. 3B, 4 mice 98 units). The loss of negative regulation of the network MFRs by NREM sleep was due to the reconfiguration of MFRs within the local CA1 network. Namely, low-firing rate units showed an increase in NREM sleep with median gain factor of +12% (p<0.0001, 50% of all the units, data not shown), while the gain factor of high-firing rate units was not affected by NREM sleep (p=0.28, data not shown). Thus, fAD mutations augmented a positive effect of NREM sleep on low-firing rate neurons, while diminishing its negative effect on high-firing rate neurons. FS neurons in the CA1 showed a similar regulation by NREM sleep: MFR reduction was prominent in WT, but lost in APP/PS1 mice (data not shown). The duration of active wake and NREM sleep states was not different between the genotypes (data now shown).

As global SWA is known to be disrupted in AD patients (Lucey et al., 2019; Mander et al., 2015) and in fAD mouse models after the onset of cognitive decline (Kent et al., 2018), we used EEG/EMG recordings to test how global SWA is affected in APP/PS1 mice before robust cognitive impairments are evident. No differences in EEG SWA (spectral power of 0.5-4 Hz) during NREM sleep were found between WT and APP/PS1 mice (FIG. 3C). Furthermore, EEG power spectra were not different for other vigilance states as well between WT and APP/PS1 mice (data not shown). In contrast to the similar global SWA in 4-5-month-old mice, EEG SWA significantly decreased in older 9-month-old APP/PS1 mice showing memory decline (p=0.002, data now shown), as expected (Kent et al., 2018). Notably, the LFP SWA measured by tetrodes in the local CA1 circuitry showed a tendency towards lower levels during NREM sleep in 4-5-month-old APP/PS1 mice, but did not reach statistical significance (p=0.14, FIG. 3D). This non-significant trend for lower SWA in CA1 LFPs represents an intermediate scale between the observed differences at the local single-neuron level and the lack of differences at the global EEG scale.

Taken together, Ca²⁺ imaging and electrophysiological data with single-cell resolution suggest that local downregulation of CA1 MFRs by NREM sleep is disrupted in early-stage APP/PS1 mice, and this homeostatic dysregulation of firing rates precedes global deterioration of slow-wave oscillations.

### EXAMPLE 4

### Loss of Neuronal Suppression During General Anesthesia in APP/PS1 Mice

We next asked whether APP/PS1 mice also display dysregulated CA1 activity under a distinct low-arousal state such as general anesthesia. We first used the volatile gas anesthetic isoflurane due to its fast kinetics. We assessed three consecutive conditions associated with distinct LFP patterns: exploration in familiar environment in active wakefulness (low-amplitude high-frequency activity), moderate anesthesia (1% isoflurane, high delta 1-4 Hz power, sporadic responses to tail-pinching) and deep anesthesia (1.5% isoflurane, burst-suppression, unresponsiveness to tail-pinching). Importantly, electrophysiological markers of anesthetic depth and respiratory rate were closely monitored and showed no differences between anesthetized WT and APP/PS1 mice (data not shown), ruling out altered respiration or pharmacokinetics as potential factors.

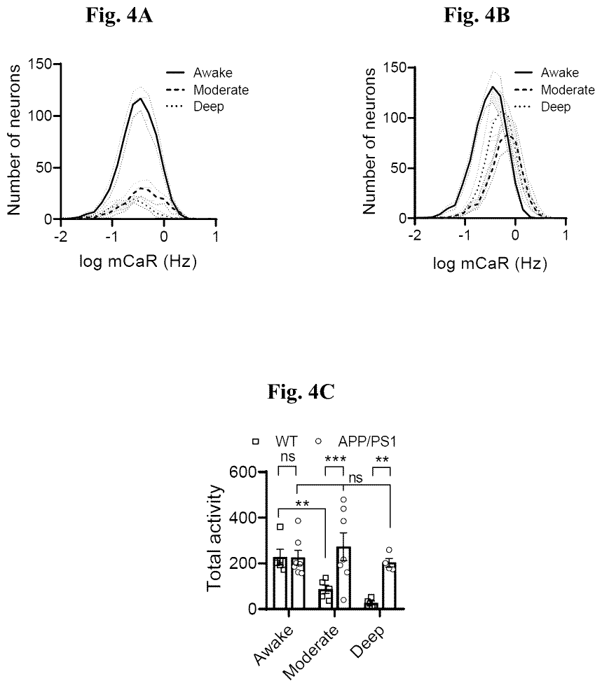

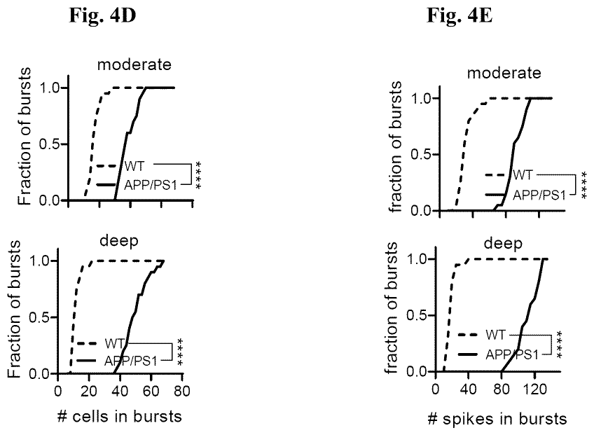

In WT mice, isoflurane caused a pronounced inhibition of CA1 population activity (FIG. 4A). The distribution of mean Ca²⁺ event rates across cells revealed suppression of total CA1 activity by ˜57% and ˜87% during moderate and deep anesthesia, respectively (average mCaR distributions of 5 mice, FIGS. 4A, 4C), mainly due to reduction in the number of active neurons in WT mice (FIG. 4A). General anesthesia had a different effect on CA1 population activity in APP/PS1 mice when compared to WT mice. Both moderate and deep anesthesia states expressed a reduced number of active neurons that was accompanied by an increase in mean Ca²⁺ event rate (p<0.05, right shift in the distribution, FIG. 4B, average of 7 mice). As a result, total activity level was maintained constant across wakefulness and anesthesia (FIGS. 4B, 4C). The loss of a typical response to general anesthetic resulted in CA1 hyperactivity in anesthetized APP/PS1 relatively to WT mice (FIG. 4C). CA1 population activity became hyper-synchronized in anesthetized APP/PS1 mice, in comparison to WTs, as reflected by more neurons participating in network bursts (FIG. 4D), more spikes evoked per network bursts (FIG. 4E) and smaller inter-burst interval under deep anesthesia (data not shown). In addition, anesthetized APP/PS1 mice displayed an augmented input-output slope at CA3-CA1 synaptic connections, in comparison to WT mice (data not shown). Thus, both, basal CA3-CA1 synaptic transmission and spontaneous CA1 spiking activity were selectively impaired in anesthesia, but not in active wakefulness, in pre-symptomatic APP/PS1 mice.

It has been recently suggested that general anesthetics is associated with a decreased number of discriminable micro-patterns of activity (microstates) at the level of local neuronal ensembles in the neocortex and hippocampus (Wenzel et al., 2019). However, whether the link between anesthesia and fewer microstates is affected by fAD mutations remains unknown. We measured microstate dynamics across anesthetic depths from the large scale Ca²⁺ imaging data in WT and APP/PS1 mice using 3 different clustering algorithms: the t-SNE/watershed segmentation, affinity propagation clustering and hierarchical clustering. While the number of discriminable microstates remained stable between different awake exploration sessions across consecutive days, it was significantly reduced across anesthetic depth in WT mice, confirming previous findings (Wenzel et al., 2019). While the number of microstates was similar between WT and APP/PS1 mice during active wakefulness, APP/PS1 mice displayed a smaller reduction in the number of microstates with increasing anesthetic depth (data not shown). In addition to the reduction in the number of microstates, the mean number of coactive neurons per frame, representing the mean size of a microstate, was also reduced under anesthesia in WT, but not in APP/PS1 mice (data not shown). Altogether, these results demonstrate that an anesthesia-mediated decrease in the number of discriminable CA1 microstates and in the number of coactive neurons is impaired in fAD model mice.

To test whether the aberrant activity of CA1 neurons in APP/PS1 mice is specific to isoflurane or a general feature of anesthesia, we recorded CA1 activity under Ketamine-Xylazine (KX) anesthesia. Ketamine is a dissociative anesthetic, inducing anesthesia mainly through blockade of N-methyl-D-aspartate (NMDA)-type glutamate receptors (Franks, 2008), and is often supplemented with xylazine, an α2-adrenergic receptor agonist. In WT mice, KX anesthesia resulted in a profound reduction in CA1 activity, but was much less efficient in APP/PS1 mice (data not shown). Finally, medetomidine (MED), another general anesthetic that selectively activates α2 adrenergic receptors, caused pronounced inhibition of CA1 activity in WT mice, while a much lower level of suppression was observed in APP/PS1 mice (data not shown). Collectively, our results indicate that APP/PS1 mice express abnormal profiles of neural activity induced by several distinct classes of anesthetics and that such changes are not due to a specific anesthetic drug.

### EXAMPLE 5

### Distinct fAD Models Express CA1 Hyperexcitability During Anesthesia

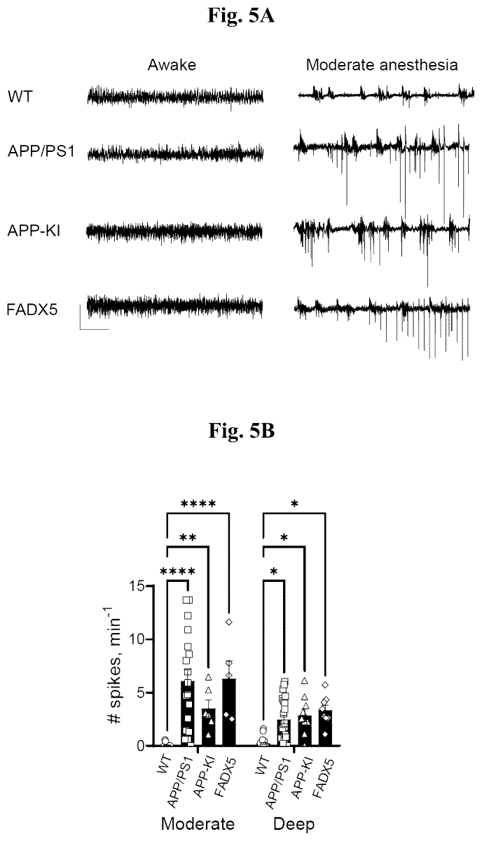

Augmented and hyper-synchronous Ca²⁺ dynamics in the soma of excitatory CA1 neurons of anesthetized fAD model mice prompted us to investigate whether these mice express silent pathological spikes, similarly to those detected in sleeping AD patients (Lam et al., 2017). To address this question, we tested how general anesthesia affects CA1 network excitability using in vivo electrophysiological recordings in the CA1 across different mouse fAD models. In addition to the APP/PS1 model, we made measurements in 5XFAD (Oakley et al., 2006, Intraneuronal β-Amyloid Aggregates, Neurodegeneration, and Neuron Loss in Transgenic Mice with Five Familial Alzheimer's Disease Mutations: Potential Factors in Amyloid Plaque Formation. The Journal of Neuroscience 26, 10129-10140), co-overexpressing mutant forms of human APP associated with the Swedish, the Florida (I716V) and the London (V717I) mutations, and App^{NL-G-F} knock-in model (Saito et al., 2014, Single App knock-in mouse models of Alzheimer's disease, Nature Neuroscience 17, 661) (APP-KI), co-expressing Swedish (KM670/671NL), Iberian (1716F) and Arctic (E693G) mutations. APP-KI mice express physiological APP levels but demonstrate an increased Aβ42/Aβ40 ratio (data not shown). Brief and abnormal high-voltage spikes were detected across all fAD models, but rarely in WT mice during isoflurane anesthesia (FIG. 5A). To further understand the relationship between anesthesia states and CA1 hyperexcitability, we quantified the frequency of abnormal spiking activity across anesthetics depth. State-specific analysis revealed higher abnormal spikes frequency for all three fAD models compared to WT, for both, moderate and deep anesthesia (FIG. 5B). Importantly, respiration rate showed no differences between all 3 fAD models and WT anesthetized mice at both, moderate and deep anesthesia (data not shown). These results indicate that pathological CA1 hyperexcitability is a common neuronal network dysfunction that emerges during low-arousal states. Moreover, the robustness of anesthesia-induced CA1 hyperexcitability in APP-KI mice indicates that fAD mutations on their own, even without APP overexpression, are sufficient to cause CA1 hyperexcitability under anesthetic-induced alerted states of arousal.

### EXAMPLE 6

### Anesthesia Induces CA1 Hyperexcitability and Impairs Spatial Working Memory and nRE-CA1 Short-Term Synaptic Plasticity in fAD Mice

Recordings of LFPs in the CA1 area of the hippocampus in early-stage, 4-5 months old APP/PS1 (APP^{SWE}/PS1ΔE9) mice revealed frequent epileptiform high-voltage spikes (3.22±0.51 per min) under general anesthesia (1.5% isoflurane, while epileptiform spiking rarely happened in wild-type (WT) mice under anesthesia (FIGS. 5A-5B). This indicates that anesthesia exposes early dysfunctions in the CA1 hippocampus of APP/PS1 mice, confirming the results of our previous study (Zarhin et al., 2022). CA1 activity is normal during active wakefulness in cognitively unimpaired, early-stage APP/PS1 mice. We hypothesized that general anesthesia would expose initial cognitive deficits in early-stage APP/PS1 mice. Working memory is a central component of cognitive function and is particularly susceptible to AD. We asked whether anesthesia produces a lasting disruption of spatial working memory in APP/PS1 mice before the appearance of the innate memory impairments.

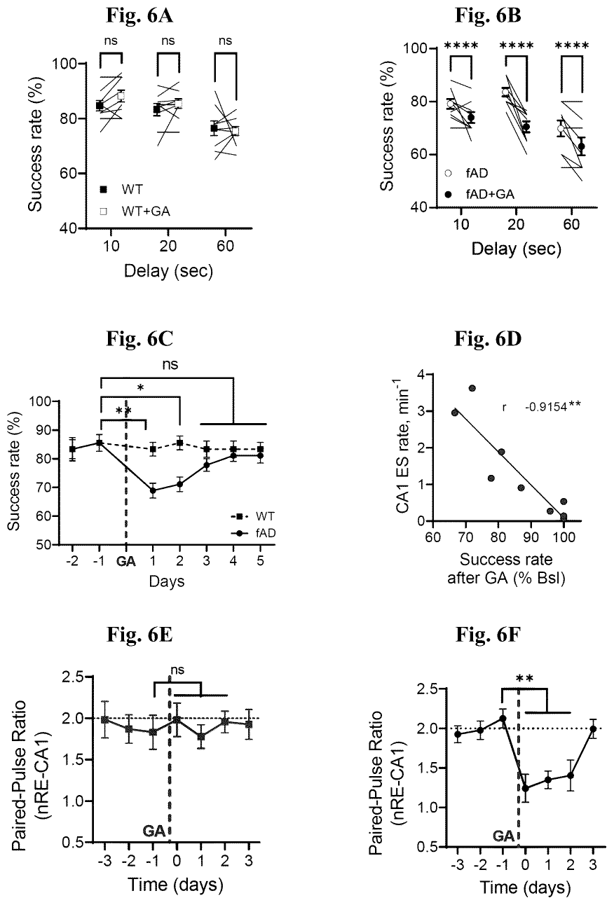

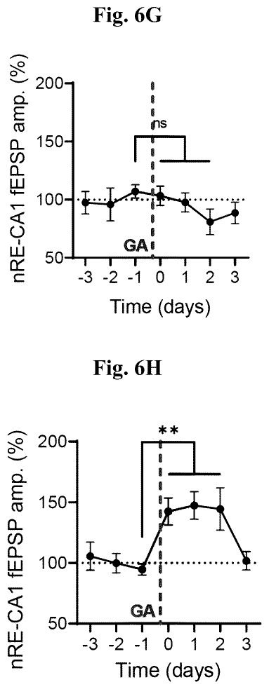

To address this question, we tested the effect of general anesthesia on the delta-maze alternation test in 4-5-month-old WT vs. APP/PS1 mice. The percentage of correct choices (alternations) was recorded for each daily session. Mice were first trained daily at a 10 sec delay between sample and choice until each mouse reached a stable success rate. All WT and APP/PS1 mice reached an asymptotic success rate of ˜80% by 8-10 days of training, indicating that they learned the task. Learning was similar between genotypes (P=0.64). Following the training, spatial working memory was tested at 10, 20 and 60 sec delays. Spatial working memory was not impaired in 4-5-month-old APP/PS1 mice, as success rates were similar between genotypes at each of the delays (data not shown). A cohort from each genotype was then anesthetized using isoflurane inhalation (1.5%, 3 hour) and performance was subsequently tested in the following days. Following anesthesia, APP/PS1 mice (FIG. 6B), but not WT (FIG. 6A), showed a significant (p<0.0001) decrease in choice accuracy in all three delay times. The impairments in spatial working memory in APP/PS1 mice were transient and there was a gradual return to the baseline performance levels over the course of 3 days following anesthesia (FIG. 6C). These experiments demonstrate that general anesthesia induces CA1 hyperexcitability and spatial working memory impairments in otherwise cognitively normal APP/PS1 mice.

To test if the decline in spatial working memory was linked to CA1 hyperexcitability exposed by anesthesia, we repeated the experiment in APP/PS1 mice implanted with LFP electrodes to record epileptiform spikes in the CA1 hippocampus. The frequency of epileptiform CA1 spikes during anesthesia strongly correlated with the degree of subsequent working memory impairment (FIG. 6D). Based on these results, we hypothesized that epileptiform spikes might represent the signal inducing maladaptive plasticity that causes working memory deficits.

Anesthesia Impairs nRE-CA1 Short-Term Synaptic Plasticity in fAD Mice.

Working memory depends on circuitry composed of the hippocampus, the mPFC and the thalamic nRE. The nRE, located in the midline limbic thalamus, is the principal thalamic input to the hippocampal CA1 region. Moreover, the thalamus is important for natural arousal regulation and is deactivated during anesthesia. We hypothesized that dysregulation of nRE-CA1 synaptic connections is involved in anesthesia-induced hyperexcitability and working memory impairments in fAD mice. To test this, we implanted a stimulating electrode into the nRE and recorded fEPSPs in CA1. Short-term synaptic facilitation is proposed to maintain working memory. We quantified the effect of anesthesia on short-term synaptic plasticity evoked by high-frequency spike bursts at the nRE-CA1 synaptic connections in awake APP/PS1 mice, in addition to basal synaptic transmission during low-frequency stimulation. Baseline measurements were made in 3 consecutive days at the same circadian time. Short-term synaptic facilitation at the nRE-CA1 synapse was normal in APP/PS1 mice before anesthesia (paired-pulse ratio 1.93±0.12 in WT and 2.09±0.1 in APP/PS1, FIGS. 6E-6F). Anesthesia led to a profound reduction in paired-pulse ratio only in APP/PS1 mice (to 1.22±0.18), which remained low for 3 days before returning to baseline levels (FIGS. 6E-6F). This suggests increased release probability in nRE-CA1 synapses of APP/PS1 mice, as basal synaptic responses to sparse stimuli were enhanced for 3 days following anesthesia in APP/PS1 (FIG. 6H) but not in WT (FIG. 6G) mice. Together, general anesthesia impaired nRE-CA1 short-term synaptic facilitation and spatial working memory, both lasting 3 days.

### EXAMPLE 7

### Downward Firing Rate Homeostasis is Impaired by fAD Mutations

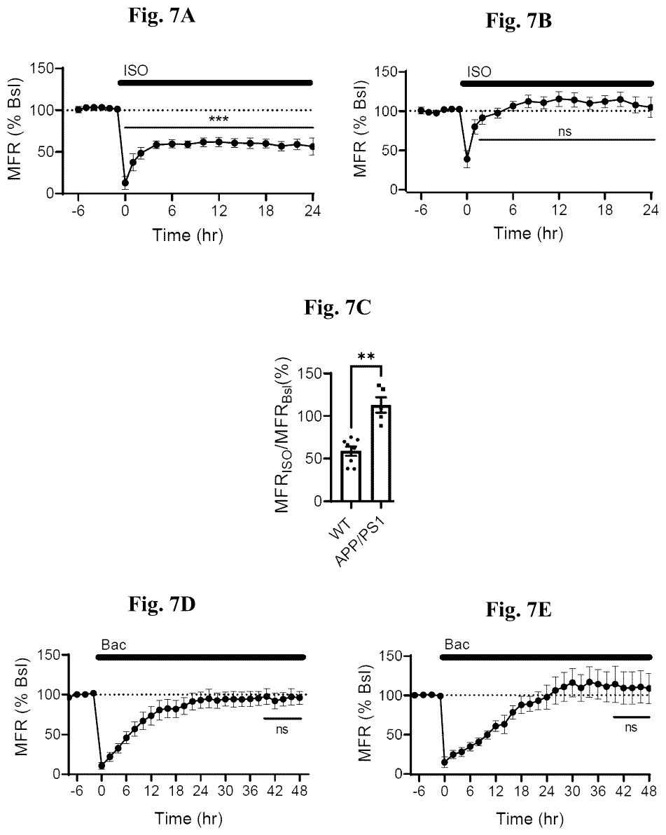

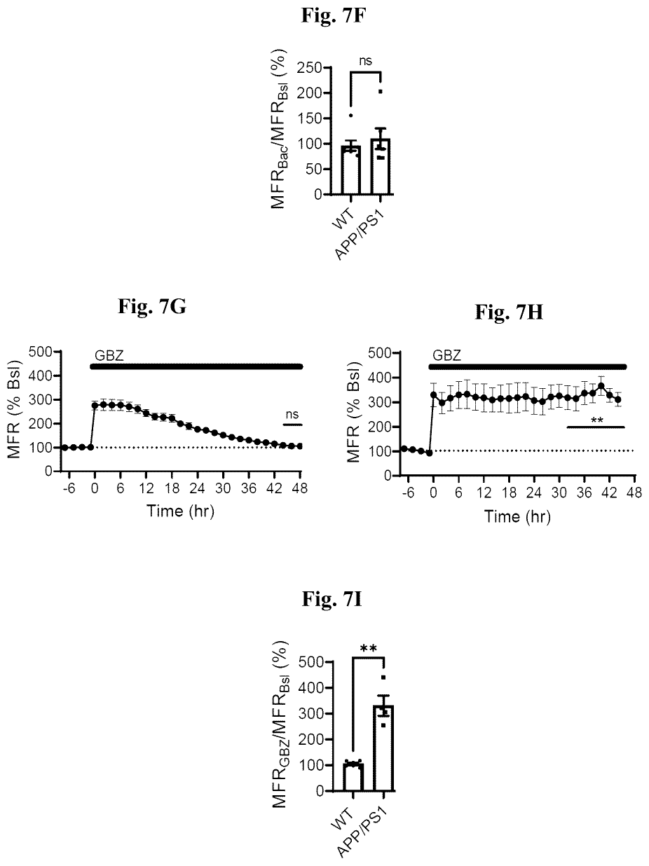

The exposure of CA1 hyperexcitability under general anesthesia may be due to impaired homeostatic regulation of MFRs. Hippocampal networks grown ex vivo on multi-electrode arrays (MEAs) have been previously established as an excellent model for dissecting the mechanisms of MFR homeostasis (Slomowitz et al., 2015; Styr et al., 2019). Therefore, we used this ex vivo platform to address the role of fAD mutations in firing rate homeostasis in response to the general anesthetic isoflurane. Spontaneous spiking activity of cultured hippocampal neurons grown on 120-channel MEAs was continuously monitored in an incubator chamber during a baseline recording period and for 24 hour following application of isoflurane. Infusion of isoflurane (1%, 40 ml/min) stably reduced MFR in WT neurons without inducing a compensatory response (FIG. 7A). This is in a striking contrast to a typical MFR renormalization to the baseline level during one day in response to inactivity, induced by GABA (B) receptor agonist baclofen (Bac) as an example (FIG. 7D). The observed compensatory response to decreased spiking activity confirms the idea that homeostatic mechanisms maintain stable circuit function by keeping network MFR around a set point. These results indicate that isoflurane disables compensatory homeostatic mechanisms, thus it constitutes a negative regulator of MFR set points in hippocampal networks. In contrast to the persistent suppression of MFRs in WT networks, isoflurane induced smaller and transient MFR decrease in APP/PS1 networks, accompanied by a fast MFR compensation to the original set point (FIG. 7B). As a result, significant hyperexcitability was observed following isoflurane application in APP/PS1 in comparison to WT networks (FIG. 7C). Notably, fAD mutations did not significantly impair homeostatic MFR response to inactivity (FIGS. 7E-7F). These results suggest that fAD mutations disrupt basic homeostatic regulation of MFR set points by isoflurane.

As isoflurane and other volatile anesthetics are known to augment inhibition by prolonging inhibitory currents mediated by GABA_{A} receptors (GABA_{A}R) in hippocampal neurons (Jones and Harrison, 1993) among other targets, we decided to test how fAD mutations affect the homeostatic response to hyperactivity imposed by GABA_{A}R blockade. As expected (Vertkin et al., 2015, GABAB receptor deficiency causes failure of neuronal homeostasis in hippocampal networks. Proc Natl Acad Sci USA 112, E3291-3299), application of a GABA_{A}R antagonist gabazine (GBZ, 30 μM) caused a fast and pronounced increase in the population firing rate that gradually declined within two days to the set-point level (FIG. 7G), despite the constant presence of gabazine. However, fAD mutations impaired MFR compensation in response to GABA_{A}R blockade (FIG. 7H), resulting in pathologically high MFR set points in APP/PS1, in comparison to WT networks (FIG. 7I). Taken together, these results demonstrate that fAD mutations result in severe impairments of homeostatic MFR regulation, and these deficits can be uncovered even in 3 weeks-old cultured hippocampal neurons in the dish, disconnected from other brain structures. Specifically, fAD mutations dysregulate the downward homeostatic mechanisms that normally keep lower MFR set points by general anesthetics or return MFRs from hyperactivity.

### EXAMPLE 8

### Dihydroorotate Dehydrogenase (DHODH) Inhibition Suppresses CA1 Hyperexcitability Under Anesthesia

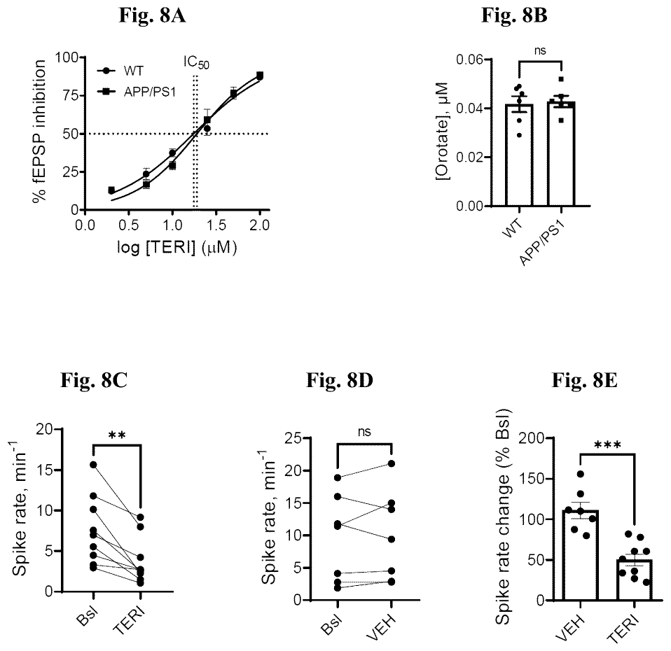

Finally, we asked whether lowering MFR set points can present an effective way to suppress CA1 hyperexcitability under anesthesia in fAD mice. Our recent work has uncovered the mitochondrial DHODH enzyme as a novel regulator of MFR set points and Ca²⁺ buffering by mitochondria during spiking activity (Styr et al., 2019). Namely, we showed that DHODH inhibition by teriflunomide (TERI) negatively regulates CA1 MFR set points and suppresses CA1 hyperexcitability in a genetic model of Dravet syndrome, one of the most intractable and severe forms of childhood epilepsy. To test whether DHODH inhibition suppresses CA1 hyperexcitability associated with fAD mutations, we first compared the dose-response of TERI on CA3-CA1 synaptic transmission in hippocampal slices prepared from WT and APP/PS1 mice. The IC₅₀ of TERI on fEPSP amplitude was not different between genotypes (FIG. 8A, 17.6 μM in WT and 19.1 μM in APP/PS1), indicating that DHODH activity is not altered by fAD mutations. Moreover, a negative regulation of MFR set points by TERI was preserved in APP/PS1 networks ex vivo (data not shown), suggesting that fAD mutations do not impair homeostatic regulation of MFR set point by DHODH. In addition, the concentration of orotate, the direct product of DHO oxidation by DHODH, was similar in the hippocampi of anesthetized WT and APP/PS1 mice (FIG. 8B), confirming that DHODH activity is not affected by fAD mutations. Based on these results, we decided to inhibit cerebral DHODH in vivo and test how it affects pathological CA1 activity in anesthetized APP/PS1 mice. As TERI does not cross blood-brain barrier efficiently, we used intracerebroventricular (ICV) infusion of TERI (27 μg in 1 μl) vs. vehicle (VEH, 1 μl) (Styr et al., 2019) to test the effect of DHODH inhibition on aberrant CA1 activity under anesthesia in APP/PS1 mice. On average, ICV infusion of TERI caused a ˜50% decrease in the rate of aberrant, high-voltage CA1 spikes in comparison to baseline recordings (FIGS. 8C, 8E). In contrast, similar amount of VEH did not affect aberrant CA1 activity (FIGS. 8D-8E). These results indicate that cerebral DHODH inhibition dampens CA1 hyperexcitability under anesthesia in APP/PS1 mice.

### EXAMPLE 9

### Inhibiting the nRE-CA1 Pathway Suppresses CA1 Hyperexcitability

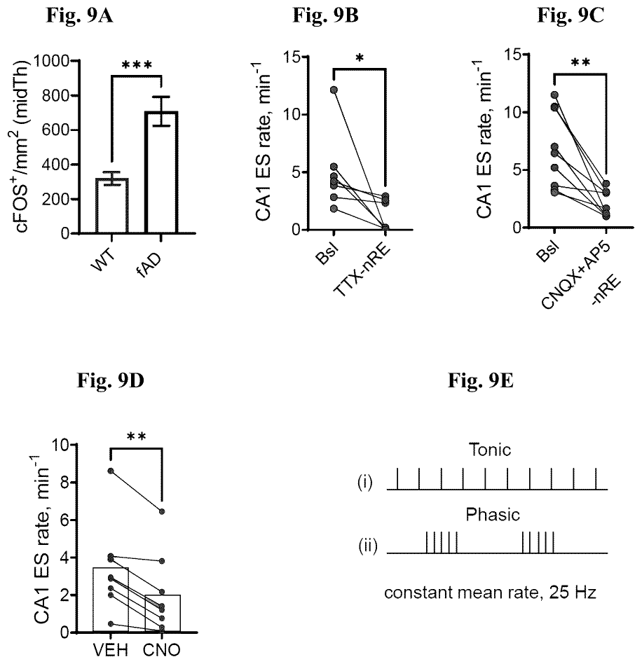

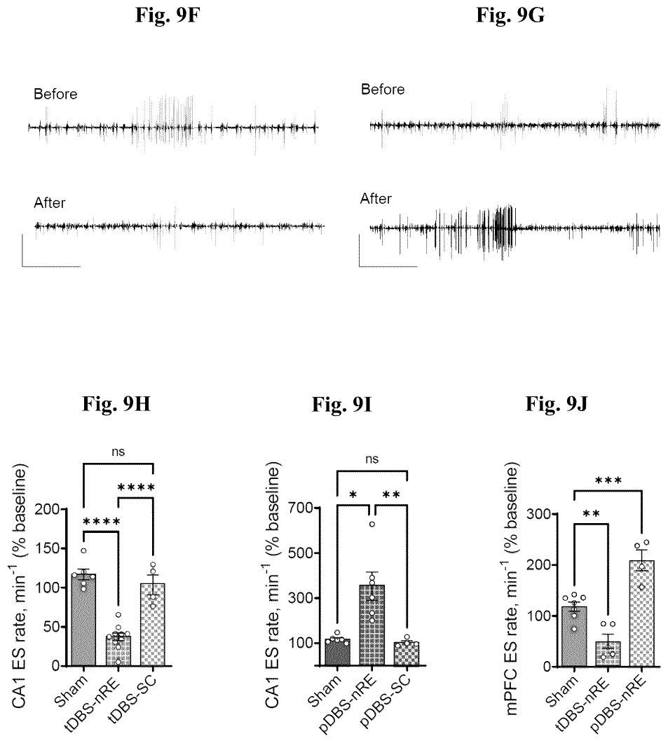

We next tested whether CA1 hyperexcitability in anesthetized APP/PS1 mice was due to synaptic inputs from the nRE. We first found that the amount of neurons expressing the activity-regulated immediate early gene cfos in the midline thalamus was significantly higher following anesthesia in APP/PS1 compared to WT mice (FIG. 9A). Next, we tested whether silencing the nRE reduces CA1 hyperexcitability under anesthesia. Pharmacological silencing of spikes in the nRE area of anesthetized APP/PS1 mice by local injection of tetrodotoxin (TTX) caused a profound decrease in the rate of epileptiform CA1 spikes in comparison to the baseline (FIG. 9B). Blocking excitatory synaptic transmission by local injections of the AMPA and NMDA receptors antagonists CNQX and AP5 to the nRE produced a similar suppressive effect on CA1 hyperexcitability (FIG. 9C). To directly test whether inhibition of the nRE-CA1 synapse can suppress anesthesia-induced CA1 hyperexcitability in fAD model mice, we combined a chemogenetic Designer Receptors Exclusively Activated by Designer Drugs (DREADD) approach (Roth, 2016) and a retrograde adeno-associated virus (Tervo et al., 2016) to specifically target nRE-CA1 synapses. The inhibitory DREADD, hM4D (Gi)-mCherry was expressed specifically in excitatory nRE neurons synapsing in CA1 by injecting APP/PS1 mice with the retro-AAV AAV2retro-CaMKIIα-iCre, in addition to a Cre-dependent hM4D (Gi)-mCherry virus in the nRE. As expected, the DREADD agonist CNO suppressed nRE-CA1 synaptic transmission (data not shown). To quantify the effect of inactivating nRE-CA1 synapses on CA1 hyperexcitability, we measured the effect of CNO on the rate of epileptiform spikes during anesthesia. On average, intraperitoneal (i.p.) injections of CNO (5 mg/kg) caused a ˜53% decrease in the rate of epileptiform CA1 spikes in comparison to the same volume of vehicle (VEH) injections (FIG. 9D). These results demonstrate that the nRE-CA1 pathway can regulate, and most likely contributes, to anesthesia-induced CA1 hyperexcitability in APP/PS1 mice.

### EXAMPLE 10

### Phasic and Tonic Stimulation of nRE Bi-Directionally Modulates CA1 and mPFC Excitability

Deep brain stimulation (DBS) has provided clinical benefit for patients with Parkinson's disease and has been proposed to present a promising therapy for AD (Jakobs et al., 2020). We thus tested whether DBS of the nRE can modulate CA1 hyperexcitability in APP/PS1 mice. The thalamus has been proposed to be a ‘choke point’ in epileptic circuits and the switch of thalamocortical neurons from a phasic to tonic firing can abort absence seizures in rodent models. Therefore, we investigated how tonic vs. phasic stimulation of the nRE modulates CA1 hyperexcitability in APP/PS1 mice. We implanted a stimulating electrode in the nRE and a recording electrode in the CA1 of APP/PS1 mice and designed two DBS protocols that had the same duration and number of stimuli, but different temporal patterns. The first protocol, termed ‘tonic DBS’ of nRE (tDBS-nRE), involved constant stimulation at 25 Hz for 400 seconds (0.2 mA amplitude, 0.5 ms pulse duration). The second protocol, termed ‘phasic DBS’ of nRE (pDBS-nRE), delivered bursts of 125 stimuli at 100 Hz, with 3.75-seconds inter-burst interval during the same 400 seconds period (schematic demonstration is presented at FIG. 9E). As controls, we used a sham condition (nRE electrode without stimulation) and stimulation of the Schaffer Collateral (SC) pathway, which is the major excitatory input to the CA1. All DBS protocols were delivered during GA.

The results showed that tDBS-nRE led to a significant ˜60% decrease in the rate of epileptiform CA1 spikes, compared to baseline, persisting for at least a week after stimulation (FIGS. 9F, 9H). In contrast, pDBS-nRE resulted in a notable ˜250% in the rate of epileptiform spikes (FIGS. 9G, 9I). Importantly, SC pathway stimulation had no significant effect on the CA1 epileptiform activity using the same tonic and phasic stimulation protocols (FIG. 9H, I). Additionally, we observed similar effects of tDBS-nRE on CA1 interictal epileptiform spikes (IESs) when using a different anesthetic (ketamine-xylazine; data not shown). These findings indicate that the impact of tDBS-nRE is not specific to isoflurane-induced hyperexcitability. Given that ketamine impairs working memory function irrespective of IESs even in wild-type rodents (Goswamee et al., 2022; Pitsikas and Boultadakis, 2009) via NMDA receptor blockade (Lisman et al., 1998), we did not continue investigating its effects on working memory in fAD mice.

nRE is the main link between the CA1 hippocampus and the mPFC. We hypothesized that anesthesia may also induce hyperexcitability of the mPFC via the nRE. Indeed, we observed high-voltage epileptiform spikes in the mPFC of anesthetized APP/PS1 mice, similar to those recorded in the CA1 (data not shown). We quantified the effects of tonic vs. phasic DBS-nRE on the rate of epileptiform spikes in the mPFC of APP/PS1 mice. Similar to our findings in CA1, tDBS-nRE suppressed, while pDBS-nRE exacerbated mPFC hyperexcitability (FIG. 9J). Altogether, these results show that nRE can bi-directionally regulate CA1 and mPFC hyperexcitability as a function of the stimulation temporal pattern in early-stage APP/PS1 mice and provide further evidence for the nRE-CA1 pathway's involvement in anesthesia-induced CA1 hyperexcitability in APP/PS1 mice, highlighting the potential of tonic DBS in treating AD-related hyperexcitability.

### EXAMPLE 11

### tDBS-nRE Restores Anesthesia-Induced Suppression of CA1 Activity

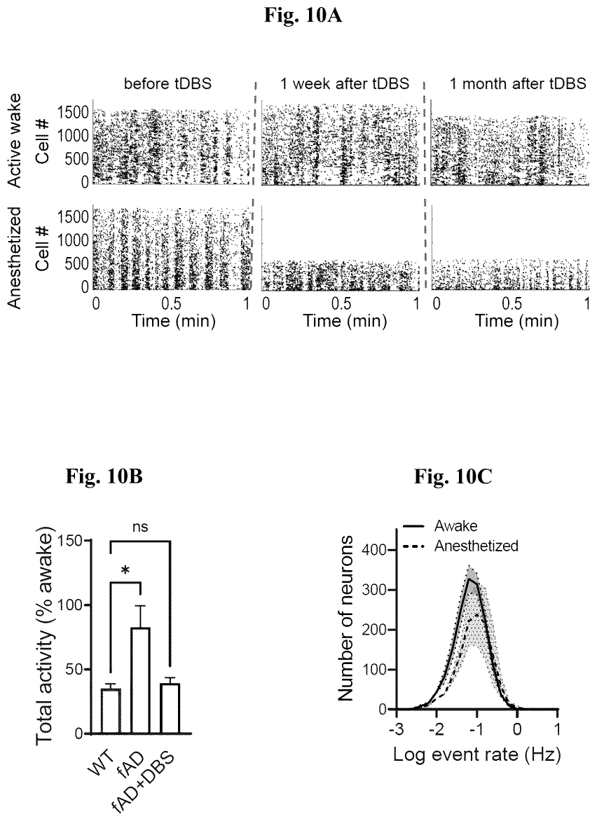

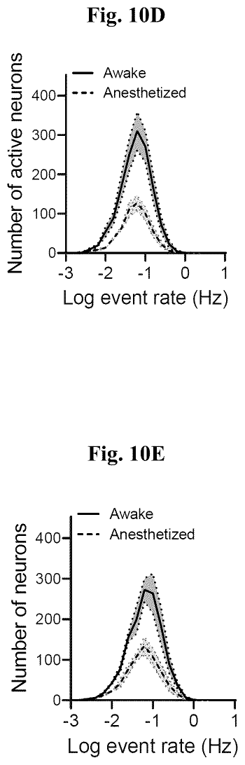

Our recent work demonstrated that general anesthesia induces dyshomeostasis of CA1 firing rates measured at a single-cell resolution in 4-5-month-old APP/PS1 mice, before the appearance of cognitive decline (Zarhin et al., 2022). In particular, we found that fAD mutations disrupted the downregulation of CA1 total activity caused by general anesthetics. tDBS-nRE restored normal CA1 excitability in APP/PS1 mice as measured by epileptiform discharges (FIG. 9E, I). To find whether tDBS-nRE can also restore normal activity downregulation in single neurons under anesthesia, we tracked Ca²⁺ dynamics as a proxy for neuronal activity within thousands of CA1 neurons using a wide-field, head-mounted miniaturized microscope. Each imaging session consisted of wake and anesthetized states in the same freely behaving APP/PS1 mice. We used a genetically encoded Ca²⁺ sensor GCaMP6f (Chen et al., 2013) expressed in excitatory CA1 pyramidal neurons under the CaMKIIα promoter. Imaging was performed at regular hours to avoid circadian effects. General anesthesia inhibited CA1 population activity in WT mice (data not shown), but not in APP/PS1 mice (FIGS. 10A—left panel, 10B, 10C). Indeed, tDBS-nRE enabled the suppression of CA1 activity by anesthesia in APP/PS1 mice a week after stimulation (FIGS. 10A—middle panel, 10D), comparable to the effect of anesthesia in WT mice (FIG. 10B). Strikingly, the effect remained stable for 1 month following the stimulation (FIGS. 10A—right panel, 10E). Interestingly, the rate of epileptiform spikes correlated with the effect of anesthesia on Ca²⁺ activity (data not shown). These results demonstrate that tDBS-nRE restores physiological down-regulation of CA1 activity by general anesthesia in APP/PS1 mice, and suggest that rescuing state-dependent regulation of CA1 firing rates contributes to the suppression of CA1 hyperexcitability by tDBS-nRE.

### EXAMPLE 12

### tDBS-nRE Prevents Anesthesia-Induced Synaptic and Memory Dysfunctions

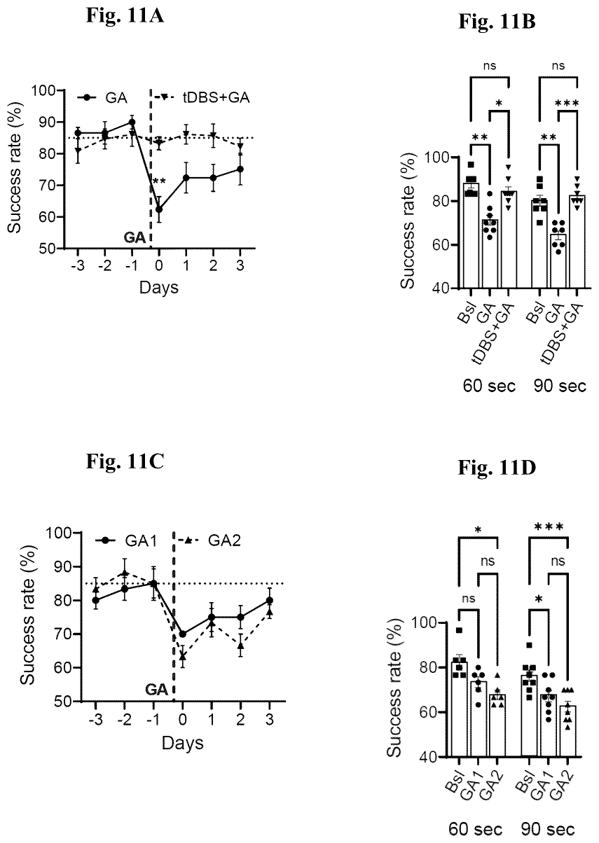

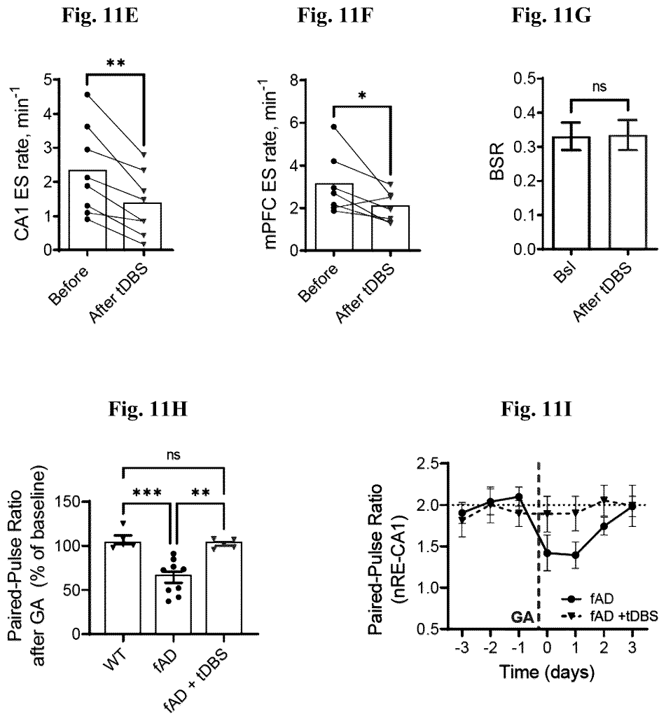

We have so far shown that nRE-CA1 short-term synaptic plasticity is impaired by anesthesia in APP/PS1 mice (FIGS. 6F-6G) and that the tDBS-nRE restores CA1 firing rates and suppresses CA1 and mPFC IESs during anesthesia in APP/PS1 mice (FIGS. 9-10). We next hypothesized that the tDBS-nRE can also restore anesthesia-induced synaptic and cognitive impairments. To test this hypothesis, we implanted APP/PS1 mice with stimulating electrodes in the nRE and LFP recording electrodes in the CA1 and mPFC. Mice were trained and tested daily in the delta maze, as previously described (Wood et al., 2000), and their performance following general anesthesia was compared to a stable baseline (SWM-T1 vs. SWM-T2). Similar to the first (un-implanted) cohort (FIGS. 6B-6C), APP/PS1 mice displayed a significant decrease in choice accuracy following anesthesia that mostly recovered after 3 days (FIGS. 11A-11B). We then tested whether tDBS-nRE promotes cognitive resilience to a second round of anesthesia in the same mice, using tDBS-nRE in awake, freely behaving mice 5 days prior to the next spatial working memory test (SWM-T3). In control (non-DBS) mice, a second session of general anesthesia led to reduced choice accuracy similar to the first anesthesia (FIGS. 11C-11D). However, tDBS-nRE prevented the reduction in choice accuracy after the second general anesthesia (SWM-T4; FIG. 11A). Mice performance was unimpaired following anesthesia in the tDBS group in comparison to their performance following anesthesia before tDBS-nRE, unlike control group, for 60 and 90 sec delay intervals (FIG. 11B). The rate of both CA1 and mPFC IESs during anesthesia was significantly lower after tDBS-nRE in the same cohort of mice (FIGS. 11E-11F). Physiological parameters of anesthetic depth, such as burst-suppression rate (FIG. 11G), heart rate, and respiratory rate (data not shown), were similar before and after tDBS. Moreover, tDBS-nRE prevented a reduction in paired-pulse ratio induced by anesthesia, maintaining it at 1.9±0.11 (FIGS. 11H-11I). These results demonstrate that tonic nRE stimulation rescues anesthesia-induced impairments of synaptic facilitation and spatial working memory in APP/PS1 mice.

### EXAMPLE 13

### tDBS-nRE Prevents Age-Dependent Memory Decline

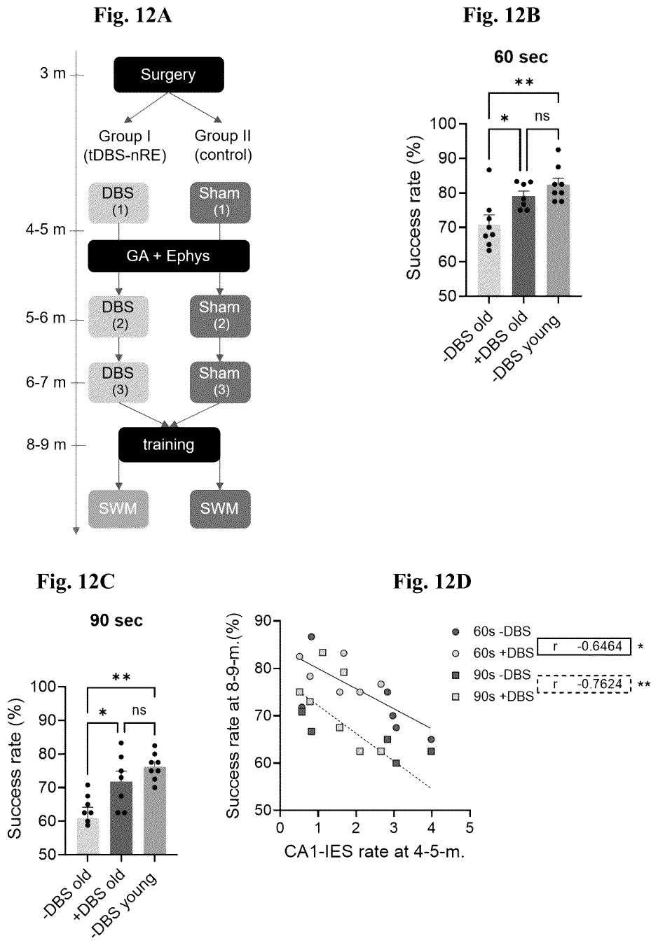

Finally, we asked whether the hyperexcitability exposed by anesthesia is solely responsible for transient memory impairments during the prodromal disease stage or if it also unmasks inherent age-related cognitive deficits at later stages. Specifically, we tested whether the thalamic nRE plays a role in the age-dependent decline of working memory during later disease stages. To address this question, we performed tDBS-nRE in young 4-5-month-old APP/PS1 mice, before the onset of working memory dysfunctions, and compared their cognitive performance to a sham control group (FIG. 12A). We implemented the stimulation protocol monthly until the mice reached an older age, characterized by robust SWM deficits. After training (data not shown), untreated 8-9-month-old APP-PS1 mice (−DBS old) exhibited significantly lower success rates in the delta maze at both 60-second and 90-second delays compared to the control group of young APP/PS1 mice (−DBS young, FIGS. 12B-12C). Notably, tDBS-nRE, initiated at 4-5 months, prevented the age-dependent decline in delta maze performance at 8-9 months in APP/PS1 mice (FIGS. 12B-12C). Furthermore, an inverse correlation was observed between the rate of IESs during anesthesia in young APP/PS1 mice and working memory at an older age for both 60-second and 90-second delays in the same mice (FIG. 12D). These results indicate that tDBS-nRE during the prodromal disease stage can effectively prevent age-dependent memory impairments.

### REFERENCES

Bakker, A., Krauss, Gregory L., Albert, Marilyn S., Speck, Caroline L., Jones, Lauren R., Stark, Craig E., Yassa, Michael A., Bassett, Susan S., Shelton, Amy L., Gallagher, M. (2012). Reduction of hippocampal hyperactivity improves cognition in amnestic mild cognitive impairment. Neuron 74, 467-474
Berger, M. et al. (2016). The effect of propofol vs. isoflurane anesthesia on human cerebrospinal fluid markers of Alzheimer's disease: results of a randomized trial. Journal of Alzheimer's disease 52(4), 1299-1310
Bubu, O.M. et al. (2017). Sleep, cognitive impairment, and Alzheimer's disease: A systematic review and meta-analysis. Sleep 40(1)
Busche, M.A., Eichhoff, G., Adelsberger, H., Abramowski, D., Wiederhold, K.H., Haass, C., Staufenbiel, M., Konnerth, A., Garaschuk, O. (2008). Clusters of hyperactive neurons near amyloid plaques in a mouse model of Alzheimer's disease. Science 321, 1686-1689
Busche, M.A., Chen, X., Henning, H.A., Reichwald, J., Staufenbiel, M., Sakmann, B., Konnerth, A. (2012). Critical role of soluble amyloid-β for early hippocampal hyperactivity in a mouse model of Alzheimer's disease. PNAS 109(22), 8740-8745
Chen, T.-W. et al. (2013). Ultrasensitive fluorescent proteins for imaging neuronal activity. Nature 499, 295-300
Eckenhoff, M.D., Roderic G., Johansson, M.D.P.D., Jonas S., Wei, M.D.P.D.H., Carnini, P.D.A., Kang, M.D.B., Wei, M.D.W., Pidikiti, M.D.R., Keller, B.S., Jason M., Eckenhoff, P.D. Maryellen F. (2004). Inhaled anesthetic enhancement of amyloid-β oligomerization and cytotoxicity. Anesthesiology 101, 703-709
Goswamee, P., Rice, R., Leggett, E., Zhang, F., Manicka, S., Porter, J.H., McQuiston, A.R. (2022). Effects of subanesthetic ketamine and (2R,6R) hydroxynorketamine on working memory and synaptic transmission in the nucleus reuniens in mice. Neuropharmacology 208, 108965
Hall, A.M., Throesch, B.T., Buckingham, S.C., Markwardt, S.J., Peng, Y., Wang, Q., Hoffman, D.A., Roberson, E.D. (2015). Tau-dependent Kv4.2 depletion and dendritic hyperexcitability in a mouse model of Alzheimer's disease. The Journal of neuroscience 35, 6221-6230
Holth, J.K., Mahan, T.E., Robinson, G.O., Rocha, A., Holtzman, D.M. (2017). Altered sleep and EEG power in the P301S Tau transgenic mouse model. Annals of Clinical and Translational Neurology 4, 180-190
Holth, J.K. et al. (2019). The sleep-wake cycle regulates brain interstitial fluid tau in mice and CSF tau in humans. Science 363 (6429), 880-884
Jakobs, M., Lee, D.J., Lozano, A.M. (2020). Modifying the progression of Alzheimer's and Parkinson's disease with deep brain stimulation. Neuropharmacology 171, 107860
Jones, M.V., Harrison, N.L. (1993). Effects of volatile anesthetics on the kinetics of inhibitory postsynaptic currents in cultured rat hippocampal neurons. J Neurophysiol, 70, 1339-1349
Ju, Y.-E.S. et al. (2017). Slow wave sleep disruption increases cerebrospinal fluid amyloid-β levels. Brain 140 (8), 2104-2111
Kang, J.E., Lim, M.M., Bateman, R.J., Lee, J.J., Smyth, L.P., Cirrito, J.R., Fujiki, N., Nishino, S., Holtzman, D.M. (2009). Amyloid-beta dynamics are regulated by orexin and the sleep-wake cycle. Science 326, 1005-1007
Kent, B.A., Strittmatter, S.M., Nygaard, H.B. (2018). Sleep and EEG power spectral analysis in three transgenic mouse models of Alzheimer's disease: APP/PS1, 3xTgAD, and Tg2576. Journal of Alzheimer's Disease 64, 1325-1336
Lam, A.D., Deck, G., Goldman, A., Eskandar, E.N., Noebels, J., Cole, A.J. (2017). Silent hippocampal seizures and spikes identified by foramen ovale electrodes in Alzheimer's disease. Nature Medicine 23, 678-680
Lisman, J.E., Fellous, J.M., Wang, X.J. (1998). A role for NMDA-receptor channels in working memory. Nat. Neurosci. 1, 273-275
Lucey, B.P. et al. (2018). Effect of sleep on overnight cerebrospinal fluid amyloid β kinetics. Annals of neurology 83, 197-204
Lucey, B.P. et al. (2019). Reduced non-rapid eye movement sleep is associated with tau pathology in early Alzheimer's disease. Science translational medicine 11(474)
Lucey, B.P. (2020). It's complicated: The relationship between sleep and Alzheimer's disease in humans. Neurobiology of Disease 144, 105031
Mander, B.A., Marks, S.M., Vogel, J.W., Rao, V., Lu, B., Saletin, J.M., Ancoli-Israel, S., Jagust, W.J., Walker, M.P. (2015). β-amyloid disrupts human NREM slow waves and related hippocampus-dependent memory consolidation. Nature Neuroscience 18, 1051-1057
Minkeviciene, R. et al. (2009). Amyloid beta-induced neuronal hyperexcitability triggers progressive epilepsy. The Journal of Neuroscience 29, 3453-3462
Musiek, E.S., Holtzman, D.M. (2016). Mechanisms linking circadian clocks, sleep, and neurodegeneration. Science 354, 1004-1008
Palop, J.J. et al. (2007). Aberrant excitatory neuronal activity and compensatory remodeling of inhibitory hippocampal circuits in mouse models of Alzheimer's disease. Neuron 55, 697-711
Palop, J.J., Mucke, L. (2009). Epilepsy and cognitive impairments in Alzheimer disease. Arch Neurol 66 (4), 435-440
Pitsikas, N., Boultadakis, A. (2009). Pre-training administration of anesthetic ketamine differentially affects rats' spatial and non-spatial recognition memory. Neuropharmacology 57, 1-7
Planel, E. et al. (2007). Anesthesia leads to tau hyperphosphorylation through inhibition of phosphatase activity by hypothermia. The Journal of Neuroscience 27, 3090-3097
Quiroz, Y.T., Budson, A.E., Celone, K., Ruiz, A., Newmark, R., Castrillón, G., Lopera, F., Stern, C.E. (2010). Hippocampal hyperactivation in presymptomatic familial Alzheimer's disease. Annals of Neurology 68, 865-875
Roh, J.H., Huang, Y., Bero, A.W., Kasten, T., Stewart, F.R., Bateman, R.J., Holtzman, D.M. (2012). Disruption of the sleep-wake cycle and diurnal fluctuation of beta-amyloid in mice with Alzheimer's disease pathology. Science translational medicine 4 (150)
Roth, B.L. (2016). DREADDs for Neuroscientists. Neuron 89, 683-694
Slomowitz, E., Styr, B., Vertkin, I., Milshtein-Parush, H., Nelken, I., Slutsky, M., Slutsky, I. (2015). Interplay between population firing stability and single neuron dynamics in hippocampal networks. Elife 4
Styr, B. et al. (2019). Mitochondrial regulation of the hippocampal firing rate set point and seizure susceptibility. Neuron 102 (5), 1009-1024
Tervo, D.G.R., Hwang, B.-Y., Viswanathan, S., Gaj, T., Lavzin, M., Ritola, K.D., Lindo, S., Michael, S., Kuleshova, E., Ojala, D., Huang, C.-C., Gerfen, C.R., Schiller, J., Dudman, J.T., Hantman, A.W., Looger, L.L., Schaffer, D.V., Karpova, A. (2016). A designer AAV variant permits efficient retrograde access to projection neurons. Neuron 92(2), 372-382
Verret, L. et al. (2012). Inhibitory interneuron deficit links altered network activity and cognitive dysfunction in Alzheimer model. Cell 149, 708-721
Vossel, K.A. et al. (2013). Seizures and epileptiform activity in the early stages of Alzheimer disease. JAMA Neurology, 1-9
Vossel, K.A. et al. (2016). Incidence and impact of subclinical epileptiform activity in Alzheimer's disease. Annals of Neurology 80, 858-870
Vutskits, L., Xie, Z. (2016). Lasting impact of general anaesthesia on the brain: mechanisms and relevance. Nature Reviews Neuroscience 17, 705
Wang, C., Holtzman, D.M. (2020). Bidirectional relationship between sleep and Alzheimer's disease: role of amyloid, tau, and other factors. Neuropsychopharmacology 45, 104-120
Watson, Brendon O., Levenstein, D., Greene, J.P., Gelinas, Jennifer N., Buzsáki, G. (2016). Network homeostasis and state dynamics of neocortical sleep. Neuron 90, 839-852
Wenzel, M., Han, S., Smith, E.H., Hoel, E., Greger, B., House, P.A., Yuste, R. (2019). Reduced repertoire of cortical microstates and neuronal ensembles in medically induced loss of consciousness. Cell Systems 8(5), 467-474
Whittington, R.A., Virág, L., Gratuze, M., Lewkowitz-Shpuntoff, H., Cheheltanan, M., Petry, F., Poitras, I., Morin, F., Planel, E. (2019). Administration of the benzodiazepine midazolam increases tau phosphorylation in the mouse brain. Neurobiology of Aging 75, 11-24
Wood, E.R., Dudchenko, P.A., Robitsek, R.J. Eichenbaum, H. (2000). Hippocampal neurons encode information about different types of memory episodes occurring in the same location. Neuron 27(3), 623-633
Xie, Z., Culley, D.J., Dong, Y., Zhang, G., Zhang, B., Moir, R.D., Frosch, M.P., Crosby, G., Tanzi, R.E. (2008). The common inhalation anesthetic isoflurane induces caspase activation and increases amyloid β-protein level in vivo. Annals of Neurology 64, 618-627
Xie, L. et al. (2013). Sleep Drives Metabolite Clearance from the Adult Brain. Science 342, 373-377
Zhang, B., Tian, M., Zheng, H., Zhen, Y., Yue, Y., Li, T., Li, S., Marcantonio, E.R., Xie, Z. (2013). Effects of anesthetic isoflurane and desflurane on human cerebrospinal fluid Aβ and τ level. Anesthesiology 119, 52-60
Zarhin, D. et al. (2022). Disrupted neural correlates of anesthesia and sleep reveal early circuit dysfunctions in Alzheimer models. Cell Rep 38(3), 110268
Zhou, P. et al. (2018). Efficient and accurate extraction of in vivo calcium signals from microendoscopic video data. eLife 7, e28728
Ziv, Y., Burns, L.D., Cocker, E.D., Hamel, E.O., Ghosh, K.K., Kitch, L.J., El Gamal, A., Schnitzer, M.J. (2013). Long-term dynamics of CA1 hippocampal place codes. Nat Neurosci 16, 264-266

1. A method of identifying and treating a subject in a process of developing Alzheimer's disease (AD) or mild cognitive impairment (MCI), said method comprising:
referring the subject for diagnostic anesthesia during which the presence of epileptiform spikes in the brain of said subject is detected, thereby identifying the subject as being in the process of developing AD or MCI, wherein said subject does not present clinical symptoms associated with AD or MCI; and
providing the identified subject with a treatment capable of attenuating or reducing cortico-hippocampal hyperactivity, thereby delaying the onset of symptoms of said AD or MCI.

2. The method of claim 1, wherein said subject is about to undergo general anesthesia.

3. The method of claim 1, wherein said subject is at risk for developing AD or MCI.

4. The method of claim 3, wherein said subject at risk for developing AD or MCI is a subject having a genetic predisposition to and/or a family history of AD or MCI, or a subject older than 60 years old.

5. The method of claim 1, wherein said epileptiform spikes are detected at the hippocampus.

6. The method of claim 5, wherein said epileptiform spikes are detected at the CA1 or at the medial prefrontal cortex (mPFC).

7. The method of claim 1, wherein detecting the presence of said epileptiform spikes in the brain of said subject during diagnostic anesthesia is performed by electroencephalogram (EEG), functional magnetic resonance imaging (fMRI), positron emission tomography (PET) scan, and/or magnetoencephalography (MEG).

8. The method of claim 1, wherein said treatment capable of attenuating or reducing cortico-hippocampal hyperactivity is selected from the group consisting of administration of an active agent capable of reducing dihydroorotate dehydrogenase (DHODH) enzyme activity in the central nervous system (CNS); administration of an antiepileptic drug (AED); and application of deep brain stimulation (DBS).

9. The method of claim 8, wherein said active agent capable of reducing DHODH enzyme activity in the CNS is a nucleic acid molecule or a small molecule.

10. The method of claim 8, wherein said treatment capable of attenuating or reducing cortico-hippocampal hyperactivity is DBS; and said DBS is applied to the thalamic nucleus reuniens (nRE), and/or said DBS is tonic DBS.

11. A method of identifying and treating a subject about to undergo general anesthesia, who is at risk for developing post-anesthesia delirium or cognitive dysfunction, said method comprising:
detecting the presence of epileptiform spikes in the brain of said subject during diagnostic anesthesia, wherein said subject does not present clinical symptoms associated with Alzheimer's disease (AD) or mild cognitive impairment (MCI); and the presence of said epileptiform spikes in the brain of said subject indicates that said subject is in a process of developing said AD or MCI and thus at risk for developing post-anesthesia delirium or cognitive dysfunction; and
providing the identified subject, prior to said general anesthesia, with a treatment capable of attenuating or reducing cortico-hippocampal hyperactivity, thereby reducing likelihood of or attenuating said post-anesthesia delirium or cognitive dysfunction.

12. The method of claim 11, further comprising identifying said subject as a subject in need of a treatment that requires general anesthesia, prior to detecting the presence of the epileptiform spikes.

13. The method of claim 11, further comprising administering general anesthesia to the subject after providing the treatment to the subject.

14. A method for reducing likelihood of or delaying the onset of clinical symptoms of Alzheimer's disease (AD) or mild cognitive impairment (MCI) in a subject in need thereof, wherein said subject does not present clinical symptoms associated with said AD or MCI, and has been identified as presenting epileptiform spikes in the brain during diagnostic anesthesia, indicating that said subject is in a process of developing said AD or MCI; and said method comprises providing said subject with a treatment capable of attenuating or reducing cortico-hippocampal hyperactivity, thereby reducing likelihood of or delaying the onset of clinical symptoms associated with said AD or MCI.

15. A method for reducing likelihood of or attenuating post-anesthesia delirium or cognitive dysfunction in a subject about to undergo general anesthesia, wherein said subject does not present clinical symptoms associated with Alzheimer's disease (AD) or mild cognitive impairment (MCI), and has been identified as presenting epileptiform spikes in the brain during diagnostic anesthesia, indicating that said subject is in a process of developing said AD or MCI, and said method comprises providing said subject, prior to said general anesthesia, with a treatment capable of attenuating or reducing cortico-hippocampal hyperactivity, thereby reducing likelihood of or attenuating said post-anesthesia delirium or cognitive dysfunction.

16. A method of treating Alzheimer's disease (AD) or mild cognitive impairment (MCI) in a subject suffering from AD or MCI, or reducing likelihood of or delaying the onset of clinical symptoms associated with said AD or MCI in an asymptomatic subject in a process of developing AD or MCI, said method comprising applying deep brain stimulation (DBS) to the thalamic nucleus reuniens (nRE) of the brain of said subject.

17. The method of claim 16, wherein said DBS is tonic DBS.

18. A method of identifying a subject in a process of developing Alzheimer's disease (AD) or mild cognitive impairment (MCI), wherein said subject does not present clinical symptoms associated with AD or MCI, said method comprising:
administering diagnostic anesthesia to the subject; and
identifying the presence or absence of epileptiform spikes in the brain of said subject during said diagnostic anesthesia by performing a technique selected from the group consisting of electroencephalogram (EEG), functional magnetic resonance imaging (fMRI), positron emission tomography (PET) scan, and/or magnetoencephalography (MEG),
wherein the presence of said epileptiform spikes indicates that the subject is in the process of developing AD or MCI.

19. A method of identifying a subject about to undergo general anesthesia, who is at risk for developing post-anesthesia delirium or cognitive dysfunction, said method comprising:
administering diagnostic anesthesia to the subject; and
identifying the presence or absence of epileptiform spikes in the brain of said subject during said diagnostic anesthesia by performing a technique selected from the group consisting of electroencephalogram (EEG), functional magnetic resonance imaging (fMRI), positron emission tomography (PET) scan, and/or magnetoencephalography (MEG),
wherein the presence of said epileptiform spikes indicates that the subject is in a process of developing AD or MCI, and thus at risk for developing post-anesthesia delirium or cognitive dysfunction.
